(19) United States
(12) Patent Application Publication
(10) Pub. No.: US 2025/0325684 A1
(43) Pub. Date: Oct. 23, 2025

(54) POTENT ANTI-CANCER CYCLOTIDES

(71) Applicant: University of Southern California, Los Angeles, CA (US)

(72) Inventors: Julio A. Camarero Palao, Los Angeles, CA (US); Dipankar Chaudhuri, Alhambra, CA (US)

(21) Appl. No.: 18/855,261

(22) PCT Filed: Mar. 3, 2023
(86) PCT No.: PCT/US2023/014535

§ 371 Date: Oct. 8, 2024

Related U.S. Application Data
(60) Provisional application No. 63/337,955, filed on May 3, 2022.

(51) Int. Cl. A61K 47/64; A61K 45/06; A61P 35/00; C07K 14/415

(52) U.S. Cl. CPC A61K 47/645; A61K 45/06; A61P 35/00; C07K 14/415; C07K 2319/10

(57) ABSTRACT

Provided herein are cyclotides and compositions containing the cyclotides and their use for the treatment of cancer.

### CROSS-REFERENCE TO RELATED APPLICATION

This application is a U.S. national phase application under 35 U.S.C. § 371 of International Application No. PCT/US2023/014535, filed Mar. 3, 2023, which in turn claims priority under 35 U.S.C. § 119(e) to U.S. Provisional Application No. 63/337,955, filed May 3, 2022, the contents of each are incorporated herein by reference in their entireties.

### STATEMENT OF GOVERNMENT SUPPORT

This invention was made with government support under R01GM113636, R35GM132072, and R01GM090323 awarded by National Institutes of Health and W81XWH-10-1-0151 awarded by Department of Defense Prostate Cancer Research Program (PCRP). The government has certain rights in the invention.

### REFERENCE TO SEQUENCE LISTING

The present application contains a Sequence Listing which has been filed electronically in XML format and is hereby incorporated by reference in its entirety. The XML copy created on Oct. 8, 2024, is named 064189-4236_SL.xml and is 23,433 bytes in size.

### BACKGROUND

Colorectal cancer (CRC) poses a major public health problem in the United States and worldwide. It ranks 3rd overall for cancer death in the world, counting for almost half (46%) of all cancer deaths in men. It is estimated that 52,980 patients will die in the US in 2021 from CRC. The lack of preventive strategies, early diagnostic methods, and effective therapies to treat recurrent colorectal and other tumors creates a pressing need to understand its pathogenesis and to identify novel therapeutic approaches. This highlights the need for more effective therapies to treat this and other cancers. This disclosure satisfies this need and provides related advantages as well.

### SUMMARY OF THE DISCLOSURE

The human double minute 2 (Hdm2) protein biological function is dysregulated in a number of human tumors, including CRC cells (2). The expression of Hdm2 is induced by p53, and Hdm2 oncoprotein functions as a negative regulator of p53. Hdm2 also interacts with retinoblastoma protein (pRB), E2F transcription factor 1 (E2F1), and RNA, suggesting that Hdm2 has also p53-independent activities. Overexpression of Hdm2 and the closely related protein HdmX (also known as Hdm4) have been observed in CRC (3), where they have been shown to promote carcinogenesis (3).

Hdm2 promotes p53 degradation through a ubiquitin-dependent pathway. In normal unstressed cells, p53 protein is unstable and present at very low levels due to its ubiquitylation by Hdm2 protein. The exact mechanism by which p53 is stabilized is unclear, although a series of post-translational modifications to itself, Hdm2 and the closely related protein HdmX, are thought to dissociate the p53-Hdm2 complex leading to increased levels of p53 (4). Like other RING domain proteins, Hdm2 functions as an adaptor protein, simultaneously binding to a cognate E2 ubiquitin-conjugating enzyme and a substrate protein, resulting in transfer of ubiquitin to the substrate and subsequent degradation by the proteasome. In this manner, p53 is constantly targeted for degradation by Hdm2/HdmX during normal non-stress conditions, as are other proteins, notably Hdm2 itself and HdmX (5).

In addition to p53, the Hdm2/HdmX E3 ligase also targets for ubiquitin-dependent degradation of other proteins like the transcription factors ATF3, FOXO3a and RUNX3; and kinases DYRK2 and HIPK2, among others, that are key for carcinogenesis in CRC (6) see FIG. 8. Overexpression of ATF3 has been shown to reduce the invasive potential of CRC cells and function as a tumor suppressor (7). FOXO proteins also act as tumor suppressors in CRC, and dysregulation and loss of FOXO3a, in particular, has been shown to be a consistent step in progression to CRC metastasis (8). FOXO3a is frequently inactivated in cancer cells by mutation of the FOXO3a gene or cytoplasmic sequestration of the protein, and its inactivation is associated with the initiation and progression of cancer (9). The transcription factor RUNX3 is also known to be an important tumor suppressor gene in several cancer types, including CRC (10). RUNX3 overexpression inhibits CRC cell migration and invasion resulting from the upregulation of matrix metalloproteinase-2 (MMP-2) and MMP-9 expression (11). The expression of kinase DYRK2 expression is significantly down-regulated in CRC tissues compared with adjacent non-tumorous tissues. Functional studies have confirmed that DYRK2 inhibits cell invasion and migration in several human CRC cell lines functioning as a tumor suppressor (12). For example, a recent study has indicated that overexpression of DYRK2 is able to inhibit colorectal cancer liver metastasis (13). DYRK2 depletion has been also shown to stabilize the telomerase reverse transcriptase protein, which results in constitutive activation of telomerase (14). Telomerase activity is high especially in cancer stem cells (15), suggesting that suppression of DYRK2 activity may be also involved in the formation of cancer stem cells. Many reports have suggested that DYRK2 is a tumor suppressor in many cancer types, including CRC (16). HIPK2 phosphorylates p53 for apoptotic activation, and is also able to activate p53-independent apoptotic pathways (17).

Although HdmX contains a RING domain that is very similar to the RING domain of Hdm2, it does not possess intrinsic E3 ubiquitin ligase activity. Instead, HdmX controls p53 abundance by modulating the levels and activity of Hdm2 (18, 19). Dimerization, mediated by the conserved C-terminal RING domains of both Hdm2 and HdmX, is critical to this activity (20). While the Hdm2 RING domains can form homodimers, heterodimers form preferentially resulting in reduced auto-ubiquitylation of Hdm2 and increased p53 ubiquitylation. Hdm2 homodimers form in the absence of HdmX resulting in self-ubiquitylation and decreased Hdm2 stability (21). The oligomeric status of HdmX in the absence of Hdm2 is uncertain although it has been suggested to be monomeric, which could contribute to its lack of E3-ligase activity. Thus, disruption of the RING-mediated Hdm2/HdmX interaction would destabilize Hdm2 and increase the levels of p53 as well as other protein targets of this E3 ligase (6). The structure of the heterodimer formed by the RING domains of Hdm2 and HdmX (22) suggests that it might be possible to obtain Hdm2/X-specific E3 ligase inhibitors by targeting the Hdm2/HdmX RING domain dimer interface rather than the primary E2 binding site that is common to many RING domain E3-ubiquitin ligases.

Cyclotides are fascinating micro-proteins (≈30 residues long) present in plants from different families including Violaceae, Rubiaceae, Cucurbitaceae, and Fabaceae families, among others. They have shown a broad array of biological activities such as protease inhibitory, anti-microbial, insecticidal, cytotoxic, anti-HIV, and hormone-like activities. They share a unique head-to-tail circular knotted topology of three disulfide bridges, with one disulfide penetrating through a macrocycle formed by the two other disulfides and inter-connecting peptide backbones, forming what is called a cystine knot topology. Cyclotides can be considered as natural combinatorial peptide framework structurally constrained by the cystine-knot scaffold and head-to-tail cyclization but in which hypermutation of essentially all residues is permitted with the exception of the strictly conserved cysteines that comprise the cystine knot. Cyclotides are characterized by possessing remarkable stability due to the presence of a backbone cyclized cystine knot topology, a small size making them readily accessible to chemical synthesis and heterologous expression, and exceedingly tolerant to sequence variations and molecular grafting. In addition, cyclotides have shown to be orally active, and capable of crossing cell membranes to efficiently target intracellular targets in vivo. Altogether, these features make the cyclotide scaffold an excellent molecular framework for the design of novel peptide-based therapeutics, making them ideal substrates for molecular grafting of biological peptide epitopes.

Applicant provides herein an isolated cyclotide polypeptide having the amino acid sequence SEQ ID NO: 1

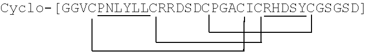

and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline). In one aspect, the isolated cyclotide polypeptide of claim 1, further comprises SEQ ID NO: 2

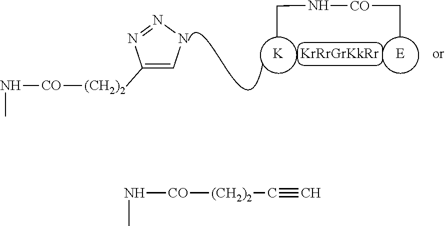

covalently attached to K at amino acid 15.

In another aspect, the isolated cyclotide polypeptide has a structure selected from:

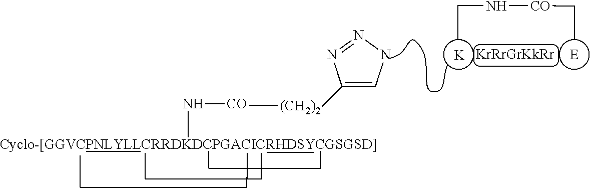

(SEQ ID NO: 2 and 3, respectively, in order of appearance) and variants thereof,
wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Ne (norleucine), Tle (terleucine), or Nva (norvaline),
wherein the MCo-52-2 cyclotide backbone to the cTAT peptide by a linker comprising —(O—CH₂CH₂—)₃, and wherein capital letters indicate an L-amino acid and lower case letters indicate D amino acids; or

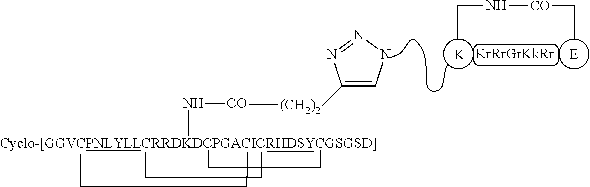

(SEQ ID NO: 2 and 3, respectively, in order of appearance) wherein the MCo-52-2 cyclotide backbone to the cTAT peptide by a linker comprising —(O—CH₂CH₂—)₃, and
wherein capital letters indicates an L-amino acid and lower case letters indicate D amino acids.

Also provided are a plurality of the isolated cyclotide polypeptides, that may be the same or different from each other.

Further provided are compositions comprising the isolated cyclotides or plurality thereof, and a carrier.

Polynucleotides encoding the cyclotide or its backbone are provided, as well as a complement of each thereof which can be provided in a pharmaceutical composition.

The isolated cyclotides and polynucleotides can further comprise a label or a purification marker.

Methods to manufacture the cyclotides are further provided herein.

The cyclotides are useful in vitro and in vivo. In one aspect, provided herein is a method to inhibit the binding of Hdm2 or HdmX to binding partners, comprising contacting Hdm2 or HdmX with an effective amount of a cyclotide as disclosed herein, thereby inhibiting the binding of Hdm2 or HdmX to its binding partner. Also provided is a method to inhibit E3 ligase activity, comprising contacting the E3 ligase with an effective amount of a cyclotide as disclosed herein, thereby inhibiting E3 ligase activity. Further provided is a method to stabilize an enzyme or peptide regulated by E3 ligase, comprising contacting the enzyme or peptide with an effective amount of a cyclotide as disclosed herein, thereby stabilizing the enzyme or peptide regulated by E3 ligase activity. The enzyme or peptides is selected from a peptide shown in FIG. 8, p53, ATF3, FOXO3a and RUNX3; and kinases DYRK2 and HIPK2. Yet further provided is a method of inhibiting the growth of a cancer cell, comprising contacting the cancer cell with an effective amount of the cyclotide of claim 3, thereby inhibiting the growth of the cancer cell, optionally wherein the cancer cell is selected from a colon cancer cell, a leukemia cell, a lung cancer cell, a small cell lung cancer cell, a pancreatic cancer cell, an ovarian cancer cell, a prostate cancer cell or a breast cancer cell. The contacting is in vitro or in vivo.

In another aspect, provided herein is a method to treat cancer or tumor in a subject in need thereof, comprising administering to the subject an effective amount of a cyclotide as disclosed herein, thereby treating the cancer, and a method to induce an anti-cancer immune response in a subject in need thereof, comprising administering to the subject an effective amount of a cyclotide as disclosed herein, thereby treating the cancer.

A kit is disclosed, the kit comprising one or more of a cyclotide, polynucleotide, vector, cell, and/or composition as disclosed herein, and optionally instructions for use.

### BRIEF DESCRIPTION OF THE DRAWING

Unless otherwise noted, all in vitro and in vivo studies reported herein were conducted using the alkyne-derivatized MCo-52-2-cTAT, even though the peptide may be identified as MCo-52-2 or 52-2 for brevity.

FIG. 1 is a schematic of “One-pot” chemical synthesis of cyclotide designed MCo-52-2 backbone, without the cTAT cell penetrating peptide attached. Figure discloses SEQ ID NO: 9.

FIG. 2: Chemoselective derivatization of cyclotide Mco-52-2-Alk with cell-penetrating peptide cTAT to give Mco-52-2-cTAT. Cyclotide Mco-52-2-cTAT was obtained by conjugation of N₃-EG₃-CONH-cTAT peptide to alkyne-derivatized cyclotide Mco-52-2-Alk using a Huisgen copper(I)-catalyzed azide-alkyne 1,3-dipolar cycloaddition (CuAAC). Mco-52-2-Alk was obtained by replacing residue Ser15 located in loop 2 by a Lys residue where the side-chain 8-amino group was acylated with 5-pentynoic acid. The thin lines connecting the Mco-52-2 cyclotide backbone to the cTAT peptide represents the linker —(—OCH₂CH₂—)₃, which is commercially available from BroadPharm (https://broadpharm.com/product/bp-21583). Capital letters indicates an L-amino acid and lower case letters indicate D-amino acids. The side-chains of the K and E amino acids are linked to each other by a —^{ε}NH^{γ}CO— linkage. Figure discloses SEQ ID NOS 2 and 2, respectively, in order of appearance.

FIGS. 3A and 3B: Pharmacokinetic profile of cyclotide MCoTI-I when dosed orally (10 mg/kg, formulated with SNAC) and through intravenous (IV, 2 mg kg) injection into male SD rats. Cyclotide was quantified from plasma using LC-MS. Animal: male SD rats. The data is represented graphically (FIG. 3A) and in a summary table showing the pharmacokinetic parameters (FIG. 3B).

FIGS. 4A-4B: Structure and E3 ligase inhibitory activity of cyclotide MCo-52-2. (FIG. 4A) Structures of the heterodimer formed by the RINGs domain of Hdm2 and HdmX and the complex of cyclotide MCo-52-2 with the RING domain of HdmX, the interacting residues in loops 1 and 5 are shown in light grey (on the left). (FIG. 4B) Inhibitory E3 ligase activity of cyclotide of Hdm2 on p53. The concentration of Hdm2 in the assay was around 1 μM as provided in the commercial kit.

FIGS. 5A and 5B: Cyclotide MCo-52-2-cTAT (represented as MCo-52-2 in the figure) activates the p53 pathway in LNCaP cells in a dose-dependent fashion. Activation of the p53 pathway requires 6 to 8 h after exposing the cells to cyclotide MCo-52-2 for 1 h (FIG. 5B). The IC₅₀ in a cell viability MTT assay for LNCaP cells treated with MCo-52-2 is 20.0±0.6 μM (FIG. 5A).

FIG. 6: Cyclotide MCo-52-2-cTAT (represented as MCo-52-2 in the figure) stabilizes transcription factors p53, FOXO3a, RUNX3, ATF3, and kinases DYRK2 and HIPK2 in HCT116 cell lines with p53-wt and -null phenotype. MCo-52-2sc is an inactive scrambled cyclotide as was used as a negative control. The IC₅₀ in a cell viability MTT assay for HCT116 and HCT116^{p53}^{_{−}}^{/}^{_{−}} cell lines treated with MCo-52-2-cTAT as described in Table 1 are 11.8±1.1 and 20.0±1.1 μM respectively.

FIG. 7: Cyclotide MCo-52-2-cTAT (designated 52-2) reduces tumor growth in two murine models of colorectal carcinoma (N=5, per group) and stabilizes p53 expression on tumor tissue as shown by western blotting analysis (right panels). No toxic effects were detected when MCo-52-2 was dosed up to 40 mg/kg three times per week. Tumor samples were also subjected to SDS-PAGE and analyzed by western blotting for p53, Hdm2, and p21. GAPDH was used as loading control in western blots.

FIG. 8 schematically shows how inhibiting Hdm2/HdmX E3 ligase activity stabilized other protein targets besides p53.

FIGS. 9A and 9B show cyclotide MCo-52-2-cTAT shows little immunogenicity in Balb/c mice. The ELISA assay was performed as follows. Micro microplate wells were coated with pmol of streptavidin and then blocked with a 5 solution of nonfat dry milk in PBS. Around of 150 pmol of the corresponding biotinylated antigen (MCo-52-2 or cTAT) were incubated with the streptavidin-coated wells. Murine IgG (m-IgG, 150 pmol) and PBS (no antigen) were used as positive and negative controls in the assay, respectively. Wells coated with antigens MCo-52-2 and cTAT were incubated with serial dilutions of sera from mice untreated and treated with cyclotide MCo-52-2-cTAT. The amount of murine IgG was detected using a goat HRP-conjugated anti-mouse IgG. FIG. 9A shows anti-MCo-52-2-cTAT IgG average titer and FIG. 9B shows anti-cTAT IgG average titer.

### DETAILED DESCRIPTION OF THE DISCLOSURE

### Definitions

This disclosure references various publications, patents and published patent specifications by an identifying citation or an Arabic number. The full citations for the disclosures referenced by an Arabic number are found immediately preceding the claims. The disclosures of these publications, patents and published patent specifications are hereby incorporated by reference into the present disclosure in their entirety to more fully describe the state of the art to which this invention pertains.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless explicitly indicated otherwise, all specified embodiments, features, and terms intend to include both the recited embodiment, feature, or term and biological equivalents thereof.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together. A recombinant polynucleotide is a polynucleotide created or replicated using techniques (chemical or using host cells) other than by a cell in its native environment.

The term “subject,” “host,” “individual,” and “patient” are as used interchangeably herein to refer to animals, typically mammalian animals or alternatively refers to a vertebrate, for example a primate, a mammal or preferably a human. As used herein, the term “animal” refers to living multi-cellular vertebrate organisms, a category that includes, for example, mammals and birds. The term “mammal” includes both human and non-human mammals. Mammals include, but are not limited to equines, canines, bovines, ovines, murines, rats, simians, humans, farm animals, sport animals and pets. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “isolated cell” generally refers to a cell that is substantially separated from other cells of a tissue. The term includes prokaryotic and eukaryotic cells.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small, secreted proteins released by immune cells that have a specific effect on the interactions and communications between the immune cells. Cytokines can be pro-inflammatory or anti-inflammatory. A non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct designed for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

HDM2 and HDMX proteins are key negative regulators of the tumor suppressor p53. Under normal conditions, p53 protein expression is maintained at a low level, whereas under stress conditions, this negative regulation is alleviated to increase the p53 level. HDM2 and HDMX are overexpressed in many cancer types, mainly in tumors with wild type p53, such as sarcomas. In addition to an inactivating mutation in the TP53 gene, HDM2 and HDMX overexpression represents another kind of p53 inactivation pathway. For additional background on these proteins see, Haronikova and Vojtesek (2018) HDM2 and HDMX proteins in human cancer, PMID 31023026 and DO: 10.14735/amko2018S63, last accessed on Feb. 28, 2023. See also https://wv.uniprot.org/uniprotkb/Q86WA4/entry, for HDM2 sequence information and https://www.ncbi.nlm.nih.gov/gene/4194 for HDMX sequence information (each last accessed on Feb. 28, 2023).

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. The degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1× SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as those described herein by a process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cells,” “host cells” or “recombinant host cells” are terms used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Innis et al. (1990) Mol. Cell Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

In one aspect, the term “equivalent” as it refers to polypeptides, proteins, or polynucleotides refers to polypeptides, oligopeptides, proteins, or polynucleotides, respectively having a sequence having a certain degree of homology or identity with the reference sequence of the polypeptides, proteins, or polynucleotides (or complement thereof when referring to polynucleotides). A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof. In one aspect, an equivalent has at least 70%, or at least 75% or at least 80%, or at least 85%, or at least 90%, or at least 95%, or at least 97%, or at least 98%, sequence identity to the reference polynucleotide or polypeptide. The term “equivalent” may also refer to a cyclotide equivalent that comprises a polypeptide that maintains a cysteine-knot scaffold and head-to-tail cyclization but in which hypermutation of essentially all residues is permitted with the exception of the strictly conserved cysteines that comprise the knot.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “oligopeptide,” “protein,” and “peptide” are used interchangeably and refer to a polymer of amino acids of any length, held together by amide bonds. Polypeptides can have any primary, secondary, tertiary, or quaternary structure and may perform any function, known or unknown. A polypeptide can comprise standard amino acids, modified amino acids, unnatural amino acids, enantiomers, and analogs thereof. If present, modifications to the amino acids can be imparted before or after assembly, synthesis, or translation of the polypeptide. A polypeptide can be further modified by conjugation with a labeling component.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. For examples of carriers, stabilizers and adjuvants, see Sambrook and Russell (2001), supra. Those skilled in the art will know many other suitable carriers for binding polynucleotides, or will be able to ascertain the same by use of routine experimentation. In one aspect of the invention, the carrier is a buffered solution such as, but not limited to, a PCR buffer solution.

A “gene delivery vehicle” is defined as any molecule that can carry inserted polynucleotides into a host cell. Examples of gene delivery vehicles are liposomes, biocompatible polymers, F including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “gene product” or alternatively a “gene expression product” refers to the RNA generated when a gene is transcribed or the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

) As used herein, a “vector” is a vehicle for transferring genetic material into a cell. Examples of such include, but are not limited to plasmids and viral vectors. A viral vector is a virus that has been modified to transduct genetic material into a cell. A plasmid vector is made by splicing a DNA construct into a plasmid. As is apparent to those of skill in the art, the appropriate regulatory elements are included in the vectors to guide replication and/or expression of the genetic material in the selected host cell.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral Vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optional contain detectable labels and are exemplified and described herein.

As used herein, the term “detectable label” intends a directly or indirectly detectable compound or composition (other than a naturally occurring polynucleotide in its natural environment) that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue.™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarry system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human.”

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

As used herein, a biological sample, or a sample, can be obtained from a subject, cell line or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

### Compositions

Provided herein is an isolated recombinant cyclotide polypeptide having the amino acid sequence SEQ ID NO: 1:

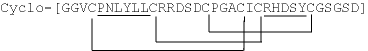

and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline), wherein “cyclo” indicates a cyclic peptide. Underlined amino acids are different from the wild-type parental protein. In one aspect, the isolated polypeptide further comprises SEQ ID NO: 2:

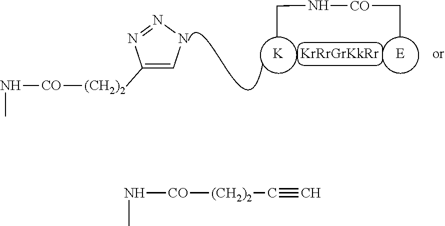

covalently attached to K at amino acid 15.

Also provided are cyclotide polypeptides having the following structures:

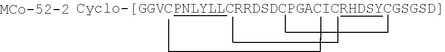

(SEQ ID NO: 1), wherein “cyclo” indicates a backbone cyclized peptide. Underlined amino acids are different from the wild-type parental protein,
or

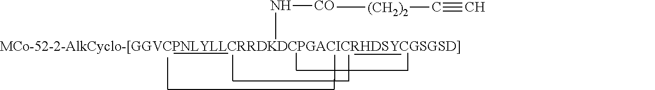

(SEQ ID NO: 4), wherein “cyclo” indicates a backbone cyclized peptide. Underlined amino acids are different from the wild-type parental protein,

### Or

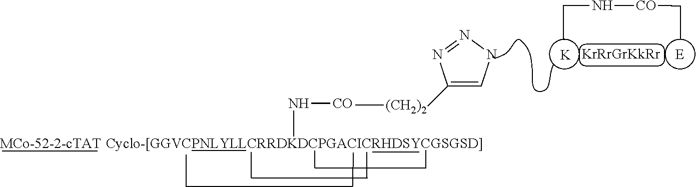

(SEQ ID NOS 2 and 3, respectively, in order of appearance), wherein the thin line connecting the MCo-52-2 cyclotide backbone to the cTAT peptide represents the linker —(O—CH₂CH₂—)₃. Capital letters indicates an L-amino acid and lower case letters indicate D amino acids. The K and E amino acids are linked to each other by a —^{ε}NH^{γ}CO— linkage. In addition, “cyclo” indicates a backbone cyclized peptide. Underlined amino acids are different from the wild-type parental protein.

Applicants note that the above noted structures are identified herein by alternate or different nomenclature.

This disclosure also provides the polynucleotides encoding the cyclotide MCo-52-2 backbone and variants thereof as described herein. The polynucleotide sequences can be determined using reverse transcription software available to those of skill in the art, see e.g., https://www.novoprolabs.com/tools/translate-protein-to-dna, last accessed on Mar. 1, 2023. An exemplary sequence comprises:

GGNGGNGTNTGYCCNAAYYTNTAYYTNYTNTGYMGNMGNGAYAARGAYTGYC CNGGNGCNTGYATHTGYMGNCAYGAYWSNTAYTGYGGNWSNWSNGGNWSNG AY (SEQ ID NO: 5). The polynucleotides can be contained within an expression vector such as a plasmid or a host cell.

In one aspect of the disclosed cyclotides and polynucleotides encoding them, they further comprise a label or purification marker.

Non-limiting exemplary labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵ Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe (III), Pr(III), Nd(III) Sm(III), Tb(III), Yb(III) Dy(III), Ho(III), Eu(II), Eu(III), or Er(III).

Further non-limiting exemplary labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a label or tag can be linked or conjugated (e.g., covalently) to the cyclotide. In various embodiments a label, such as a radionuclide or metal or metal oxide can be bound or conjugated to the agent, either directly or indirectly. A linker or an intermediary functional group can be used to link the molecule to a label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Further disclosed are a plurality of cyclotides as disclosed herein, wherein the amino acid sequences of the plurality are the same or different from each other.

Further provided are compositions comprising, or consisting essentially of, or consisting of, the cyclotides or the plurality of cyclotides as disclosed herein, and a carrier, such as for example, a pharmaceutically acceptable carrier. The compositions can further comprises an additional anti-cancer therapy. Non-limiting examples of such are disclosed herein and known in the art.

Also provided are isolated polynucleotides encoding a cyclotide as disclosed herein, and complements thereof. The isolated polynucleotides can further comprise a label or a purification marker. Examples of labels, tags and markers are disclosed above and incorporated herein by reference.

The polynucleotides can be contained within a vector for expression or replication, that are operably linked to elements for expression and replication of the polynucleotides, such as promoters and/or enhancer elements, suitable for the host cell system. Thus, further provided isolated cells comprising the polynucleotides and/or vectors as described herein. The host cells can be eukaryotic cells or a prokaryotic cells.

As noted above, compositions are further provided herein. The compositions can comprise a carrier and one or more of a cyclotide, a plurality of cyclotides, or a polynucleotide encoding same, a vector containing the polynucleotide or a host cell containing one or more of the cyclotide, the polynucleotide or vector. The carriers can be one or more of a solid support or a pharmaceutically acceptable carrier. The compositions can further comprise an adjuvant or other components suitable for administrations as vaccines or therapies. In one aspect, the compositions are formulated with one or more pharmaceutically acceptable excipients, diluents, carriers and/or adjuvants. In addition, embodiments of the compositions of the present disclosure include one or more of a cyclotide, a plurality of cyclotides, an isolated polynucleotide of the disclosure, a vector of the disclosure, an isolated host cell of the disclosure, formulated with one or more pharmaceutically acceptable auxiliary substances.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The pharmaceutical compositions can be formulated into preparations for administration in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other anticancer agents. For intravenous administration, suitable carriers include physiological saline, or phosphate buffered saline (PBS). In all cases, a composition for parenteral administration must be sterile and should be fluid to the extent that easy syringeability exists.

Aerosol formulations provided by the disclosure can be administered via inhalation and can be propellant or non-propellant based. For example, embodiments of the pharmaceutical formulations of the disclosure comprise a peptide of the disclosure formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself (e.g. by an elastic bladder)).

Suppositories of the disclosure can be prepared by mixing a compound of the disclosure with any of a variety of bases such as emulsifying bases or water-soluble bases. Embodiments of this pharmaceutical formulation of a compound of the disclosure can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds of the disclosure. Similarly, unit dosage forms for injection or intravenous administration may comprise a compound of the disclosure in a composition as a solution in sterile water, normal saline or another pharmaceutically acceptable carrier.

Embodiments of the pharmaceutical formulations of the disclosure include those in which one or more of an isolated polypeptide of the disclosure, an isolated polynucleotide of the disclosure, a vector of the disclosure, an isolated host cell of the disclosure, or an antibody of the disclosure is formulated in an injectable composition. Injectable pharmaceutical formulations of the disclosure are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations of the disclosure.

In an embodiment, one or more of an isolated polypeptide of the disclosure, an isolated polynucleotide of the disclosure, a gene delivery vehicle or vector of the disclosure, or an isolated host cell of the disclosure is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound of the disclosure can be accomplished using any of a variety of refillable, pump systems. Pumps provide consistent, controlled release over time. In some embodiments, a compound of the disclosure is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or an osmotic bursting matrix, e.g., where the drug is incorporated into a polymer and the polymer provides for the release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a peptide of the disclosure are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. After administration, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the cyclotide (as well as combination compositions) is delivered in a controlled release system. For example, the cyclotide of the disclosure may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the lung, requiring only a fraction of the systemic dose.

In another embodiment, the compositions of the present disclosure (as well as combination compositions separately or together) include those formed by impregnation of a peptide described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The compositions can comprise an additional anticancer agent, such as a chemotherapeutic or immunotherapy. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

Further provided are methods to make the cyclotides by expressing a polynucleotide encoding the polynucleotide encoding the cyclotide in a host cell, under conditions to express the polynucleotide and optionally purifying the cyclotide polypeptide. The cyclotide polypeptides can also be chemically modified as disclosed herein. Thus, this disclosure also provides the polynucleotides encoding the cyclotide MCo-52-2 backbone and variants thereof. The polynucleotide sequences can be determined using reverse transcription software available to those of skill in the art, see e.g., https://www.novoprolabs.com/tools/translate-protein-to-dna, last accessed on Mar. 1, 2023. An exemplary sequence comprises:

(SEQ ID NO: 5)
GGNGGNGTNTGYCCNAAYYTNTAYYTNYTNTGYMGNMGNGAYAARGAYT
GYCCNGGNGCNTGYATHTGYMGNCAYGAYWSNTAYTGYGGNWSNWSNGG
NWSNGAY.

In some cases, the cyclotide with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;
B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the cyclotide described herein is administered in combination with a radiation therapy.

### Dosage and Dosage Formulations

In some embodiments, the compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the cyclotide alone or in combination with the additional therapeutic agent and compositions containing the same can be affected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Therapeutic Methods

Further provided herein is a method to inhibit the binding of Hdm2 (also identified herein as HDM2) or HdmX (also identified herein as HDMX) to its binding partner, comprising, or consisting essentially of, or yet consisting of contacting Hdm2 or HdmX with an effective amount of the cyclotide, a plurality of cyclotides, or a composition as described herein to a cell containing Hdm2 or HdmX, thereby inhibiting the binding of Hdm2 or HdmX to its binding partner. In one aspect, the ring domains of Hdm2 or HdmX are inhibited. In one aspect, the binding partner of Hdm2 is selected from p53, Hdm2 or HdmX. Alternatively, the binding partner of HdmX is Hdm2. In one aspect the cyclotide is or comprises MCo-52-2-cTAT. The contacting can be in vitro or in vivo, in a cell free system, a cell system or in a subject.

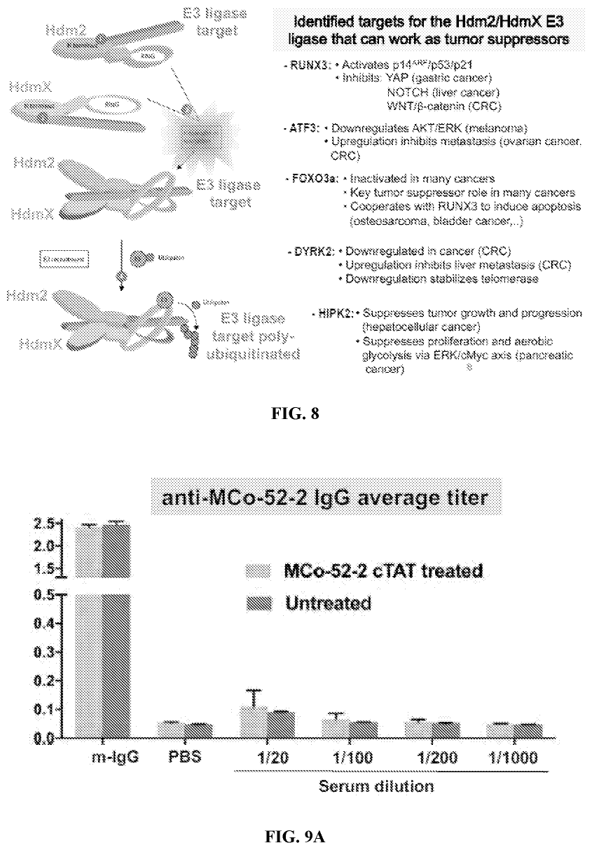

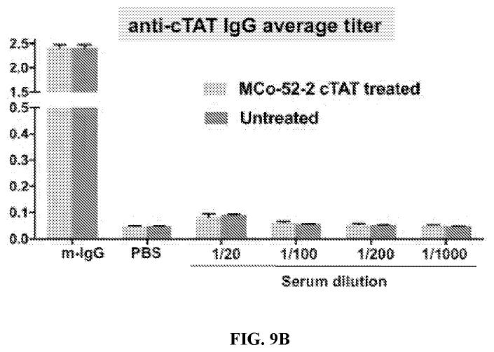

Also provided herein is a method to inhibit E3 ligase activity, comprising, or consisting essentially of, or yet further consisting of contacting the E3 ligase with an effective amount of the cyclotide of this disclosure thereby inhibiting E3 ligase activity. As shown in FIG. 8 and incorporated herein by reference, inhibiting E3 ligase activity also stabilizes other proteins/enzymes identified in the figure, e.g., ATF3, FOXO3a and RUNX3; and kinases DYRK2 and HIPK2. In this aspect, the term “stabilizes” indicates a reduction or eradication of the degradation of the protein or enzyme. In one aspect the cyclotide is or comprises MCo-52-2-cTAT. The contacting can be in vitro or in vivo, in a cell-free system, a cell system or in a subject.

Thus, this disclosure also provides a method to stabilize an enzyme or protein that is regulated by the E3 ligase activity, examples of such are shown in FIG. 8 and incorporated herein by reference, the method comprising, or consisting essentially of, or yet further consisting of contacting the enzyme or protein with an effective amount of the cyclotide of this disclosure thereby stabilizing the enzyme or protein. Examples of enzymes or proteins include for example, p53, ATF3, FOXO3a and RUNX3; and kinases DYRK2 and HIPK2. In this aspect, the term “stabilizes” indicates a reduction or eradication of the degradation of the protein or enzyme. In one aspect the cyclotide is or comprises MCo-52-2-cTAT. The contacting can be in vitro or in vivo, in a cell free system, a cell system or in a subject.

In one aspect, the cyclotide or plurality of cyclotides have the sequence or structure selected from the group of:

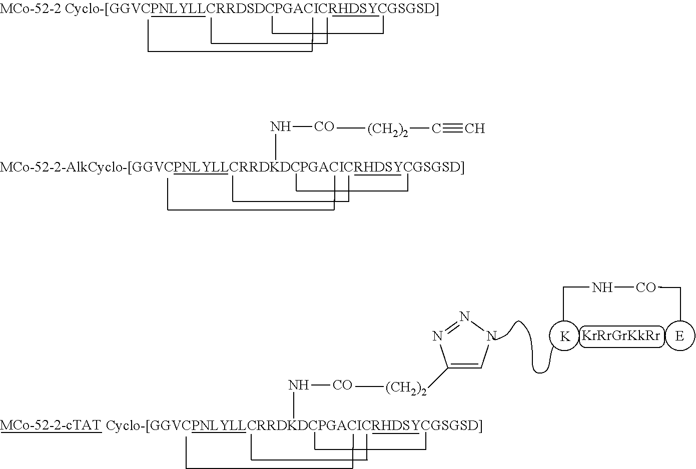

(SEQ ID NOS 1, 4, 2 and 3, respectively, in order of appearance) and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline) and, wherein the thin line connecting the MCo-52-2 cyclotide backbone to the cTAT peptide represents the linker —(O—CH₂CH₂—)₃. Capital letters indicates an L-amino acid and lower case letters indicate D amino acids. The K and E amino acids are linked to each other by a —^{ε}NH^{γ}CO— linkage. In addition, “cyclo” indicates a backbone cyclized peptide. Underlined amino acids are different from the wild-type parental protein.

For each of the above methods, the contacting can be in vitro, in vivo or ex vivo. The cells can be deficient in a tumor suppressor gene or protein, e.g., p53 gene or protein. Examples of such cells are cancer or tumor cells, such as liquid or solid tumors. Non-limiting examples include a colon cancer cell, a leukemia cell, a lung cancer cell, a small cell lung cancer cell, a pancreatic cancer cell, an ovarian cancer cell, a prostate cancer cell, a colorectal cancer cell, a rectal cancer cell or a breast cancer cell that may be a primary cancer cell or a metastatic cancer cell. When practiced in vivo, the method is useful to screen for new therapies or personalized therapies. When practiced in vivo in an animal, the method is a useful therapeutic for the animal or in a laboratory animal to test new therapies. When practiced in a human, the method is a useful therapeutic.

Further disclosed herein are methods for inducing an immune response in a subject consisting essentially of, or yet further consisting administering an effective amount of the cyclotide, plurality of cyclotides or compositions as disclosed herein, to the subject. In one aspect, the cyclotide or plurality of cyclotides have the sequence or structure selected from:

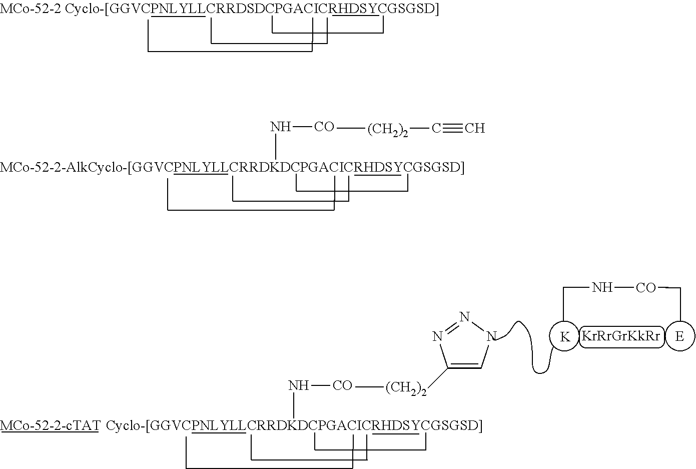

(SEQ ID NOS 1, 4, 2, and 3, respectively, in order of appearance) and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline), wherein the thin line connecting the MCo-52-2 cyclotide backbone to the cTAT peptide represents the linker —(O—CH₂CH₂—)₃. Capital letters indicates an L-amino acid and lower case letters indicate D amino acids. The K and E amino acids are linked to each other by a —^{ε}NH^{γ}CO— linkage. In addition, “cyclo” indicates a backbone cyclized peptide. Underlined amino acids are different from the wild-type parental protein.

Further provided herein is a method of inhibiting the growth of a cancer cell, comprising contacting the cancer cell with an effective amount of a cyclotide, the plurality or a composition of this disclosure, thereby inhibiting the growth of the cancer cell in a subject in need thereof, optionally wherein the cancer cell is selected from a colon cancer cell, a leukemia cell, a lung cancer cell, a small cell lung cancer cell, a pancreatic cancer cell, an ovarian cancer cell, a prostate cancer cell, colorectal cancer cell, rectal cancer cell, or a breast cancer cell that may be a primary cancer cell or a metastatic cancer cell. The cancer cells can be deficient in a tumor suppressor gene or protein, e.g., p53 gene or protein.

In one aspect, the cyclotide or plurality of cyclotides have the sequence selected from:

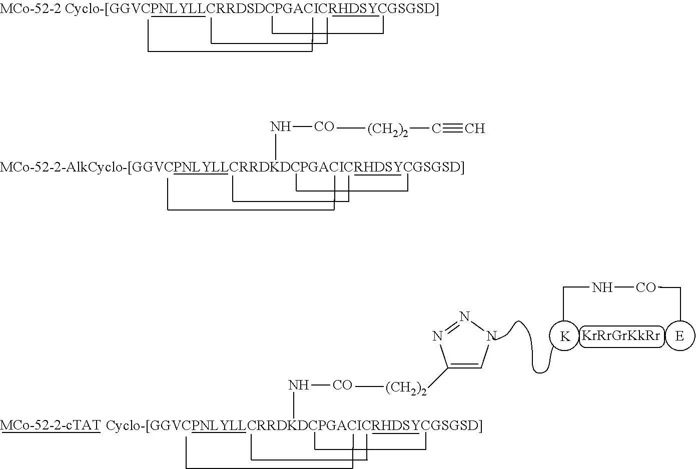

(SEQ ID NOS 1, 4, 2, and 3, respectively, in order of appearance) and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline) and wherein the thin line connecting the MCo-52-2 cyclotide backbone to the cTAT peptide represents the linker —(O—CH₂CH₂—)₃. Capital letters indicates an L-amino acid and lower case letters indicate D amino acids. The K and E amino acids are linked to each other by a —^{ε}NH^{γ}CO— linkage. In addition, “cyclo” indicates a a backbone cyclized peptide. Underlined amino acids are different from the wild-type parental protein.

Yet further provided is a method of inhibiting the growth of a tumor or cancer, or treating a cancer or tumor in a subject in need thereof, comprising contacting the cancer cell with an effective amount of the cyclotide, the plurality or a composition of this disclosure, thereby inhibiting the growth of the cancer cell inhibiting the growth of a tumor or cancer, or treating a cancer or tumor. The cancer cells can be deficient in a tumor suppressor gene or protein, e.g., p53 gene or protein. Non-limiting examples of cancer or tumors include a colon cancer, a leukemia, a lung cancer, a small cell lung cancer, a pancreatic cancer, an ovarian cancer, a prostate cancer or a breast cancer.

In one aspect, the cyclotide or plurality of cyclotides have the sequence selected from:

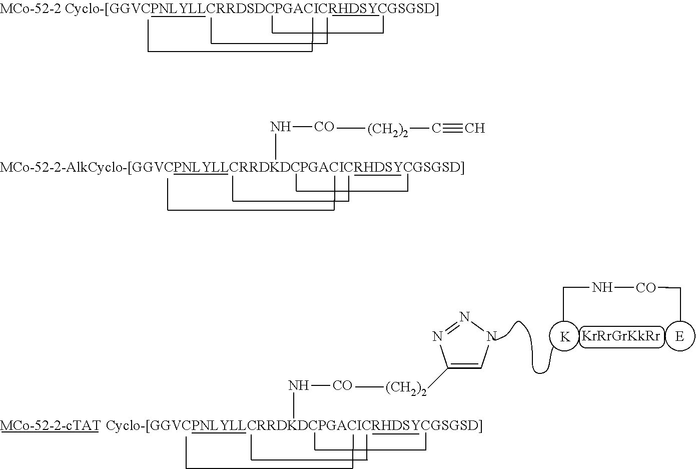

(SEQ ID NOS 1, 4, 2, and 3, respectively, in order of appearance) and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline) wherein the thin line connecting the MCo-52-2 cyclotide backbone to the cTAT peptide represents the linker —(O—CH₂CH₂—)₃. Capital letters indicates an L-amino acid and lower case letters indicate D amino acids. The K and E amino acids are linked to each other by a —^{ε}NH^{γ}CO— linkage. In addition, “cyclo” indicates a backbone cyclized peptide. Underlined amino acids are different from the wild-type parental protein.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary lung cancer or breast cancer. In some embodiments, the cancer metastatic melanoma or metastatic triple negative breast cancer. The therapy can be administered as a first line, second line, third line, fourth line or fifth line therapy.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

The methods and compositions disclosed herein may further comprise or alternatively consist essentially of, or yet further consists of administering to the subject an anti-tumor therapy other than the cyclotide or plurality disclosed herein. In some embodiments, anti-tumor therapy may include different cancer therapy or tumor resection. The additional therapeutic can be combined in the same composition or separately administered.

In some embodiments, the nanoparticle and/or composition are provided to prevent the symptoms of cancer from occurring in a subject that is predisposed or does not yet display symptoms of the cancer or to prevent a recurrence.

In some embodiments, the cyclotide or plurality may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, any of the cyclotide or plurality, are administered to the subject at least 1, 2, 3, 4, 5, 6, 7, 8, 9, or 10 times a day. In some embodiments, any of the cyclotide or plurality or compositions disclosed herein are administered to the subject at least 1, 2, 3, 4, 5, 6, 7, 8, 9, 10, 11, 12, 13, 14, 15, 16, 17, 18, 19, or 20 times a week. The cancer cells can be deficient in a tumor suppressor gene or protein, e.g., p53 gene or protein. Plurality or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the cyclotide or plurality or compositions disclosed herein are administered to the subject at least every 1, 2, 3, 4, 5, 6, 7, 8, 9, or 10 days. In some embodiments, any of the cyclotide or plurality or compositions disclosed herein are administered to the subject at least every 1, 2, 3, 4, 5, 6, 7, 8, 9, 10, 11, 12, 13, or 14 weeks. In some embodiments, any of the cyclotide or plurality or compositions disclosed herein are administered to the subject for a period of at least 1, 2, 3, 4, 5, 6, 7, 8, 9, or 10 days. In some embodiments, any of the cyclotide or plurality or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the cyclotide or plurality or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the method and compositions provided herein, comprising, or alternatively consisting essentially of, or yet further consisting inhibiting metastatic potential of the cancer, reduction in tumor size, a reduction in tumor burden, longer progression free survival, or longer overall survival of the subject.

In one aspect, the methods or compositions further comprise administration of an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the cyclotide or plurality or compositions with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, TLR1, TLR2, TLR3, TLR4, TLR5, TLR6, TLR7, TLR8, or TLR9. In some cases, the TLR ligand comprise, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the cyclotide or plurality or compositions described herein is administered in combination with a radiation therapy.

### Kits

In one particular aspect, the present disclosure provides kits for performing the methods of this disclosure as well as instructions for carrying out the methods of the present disclosure. The kit comprises, or alternatively consists essentially of, or yet further consists of one or more of cyclotide or plurality or compositions, polynucleotide, vector and/or host cell of this disclosure and instructions for use. In a further aspect, the instruction for use provide directions to conduct any of the methods disclosed herein.

The kits are useful for detecting the presence of cancer such as colon cancer or lung cancer in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Experiment No. 1

Materials and instrumentation. Analytical HPLC was performed on a HP1100 series instrument with 220 nm and 280 nm detection using a Vydac C18 column (5 μm, 4.6×150 mm) at a flow rate of 1 mL/min. Semipreparative HPLC was performed on a Waters Delta Prep system fitted with a Waters 2487 Ultraviolet-Visible (UV-vis) detector using a Vydac C18 column (15-20 μm, 10×250 mm) at a flow rate of 5 mL/min. All runs used linear gradients of 0.1% aqueous trifluoroacetic acid (TFA, solvent A) vs. 0.1% TFA, 90% acetonitrile in H₂O (solvent B). UV-vis spectroscopy was carried out on an Agilent 8453 diode array spectrophotometer, and fluorescence analysis on a Flurolog-3 spectrofluorimeter (Horiba Scientific). Electrospray mass spectrometry (ES-MS) analysis was routinely applied to all cyclized peptides. ES-MS was performed on an Applied Biosystems API 3000 triple quadrupole electrospray mass spectrometer using Analyst 1.4.2. LC-MS was performed on a HP1100 HPLC/API-3000 system using a multiple reaction monitoring (MRM) mode. Calculated masses were obtained by using ProMac v1.5.3. Protein samples were analyzed by SDS-PAGE. Samples were run on Invitrogen (Carlsbad) 4-20% Tris-Glycine Gels. The gels were then stained with Pierce (Rockford) Gelcode Blue, photographed/digitized using a Kodak (Rochester) EDAS 290, and quantified using NIH Image-J software (http://rsb.info.nih.gov/ij/). DNA sequencing was performed by the DNA Sequencing and Genetic Analysis Core Facility at the University of Southern California using an ABI 3730 DNA sequencer, and the sequence data was analyzed with DNAStar Lasergene v5.5.2. All chemicals were obtained from Sigma-Aldrich unless otherwise indicated. Nutlin-3 was purchased from sellechchem.com (www.sellechchem.com).

Preparation of Fmoc-Tyr(tBu)-F. Fmoc-Tyr(tBu)-F was prepared using diethylaminosulfur trifluoride (DAST) (C. Kaduk et al., Lett. Pept. Sci.2 (1997)) and quickly used afterwards. To a stirred solution of the Fmoc-amino acid (10 mmol) in 60 mL of dry dichloromethane (DCM), under an argon atmosphere, 805 μL (10 mmol) of pyridine (dry) were added at room temperature followed by dropwise addition of 1.57 mL (12 mmol) of DAST. After stirring for 20 min, the mixture was extracted three times with 150 mL of ice water and the combined organic layers were dried over MgSO₄ and molecular sieves (10 Å). The solvent was removed in vacuo at room temperature. Recrystallization or precipitation from DCM/n-hexane gave the Fmoc-amino acid fluoride.

Loading of 4-sulfamylbutyryl AM resin with Fmoc-Tyr(tBu)-F. Loading of the first residue was accomplished using Fmoc-Tyr(tBu)-F according to standard protocol (R. Ingenito et al., Org. Lett. 4 (2002)). Briefly, 4-sulfamylbutyryl AM resin (420 mg, 0.33 mmol) (Novabiochem) was swollen for 20 minutes with dry DCM and then drained. A solution of Fmoc-Tyr(tBu)-F (≈461 mg, 1 mmol) in dry DCM (2 mL) and di-isopropylethylamine (DIEA) (180 μL, 1 mmol) was added to the drained resin and reacted at 25° C. for 1 h. The resin was washed with dry DCM (5×5 mL), dried and kept at −20° C. until use.

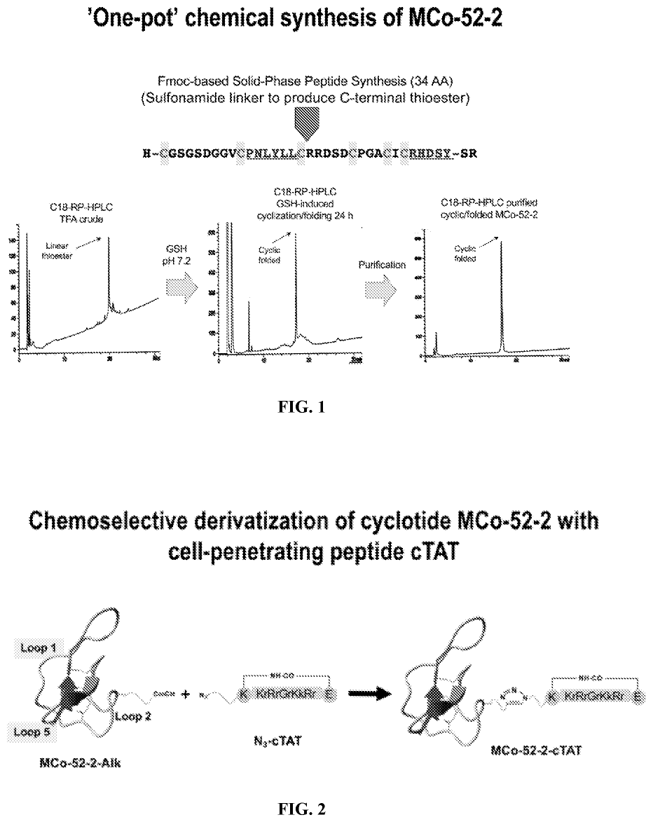

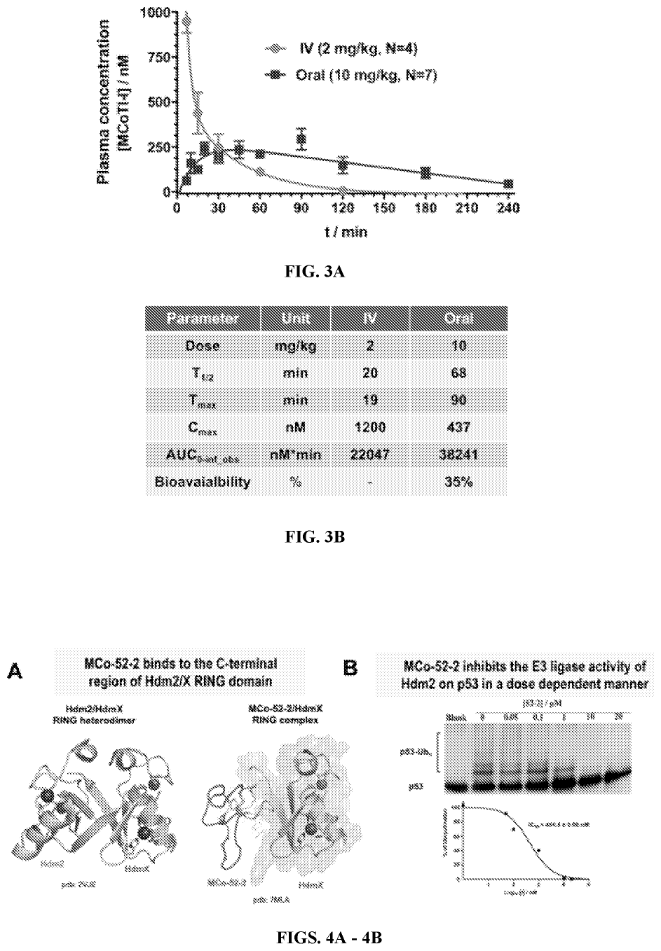

Chemical synthesis of MCo-52-2 cyclotides. Solid-phase synthesis was carried out on an automatic peptide synthesizer ABI433A (Applied Biosystems) using the Fast-Fmoc chemistry with 2-(1H-benzotriazol-1-yl)-1,1,3,3-tetramethyluronium hexafluorophosphate (HBTU) activation protocol at 0.1 mmole scale on a Fmoc-Tyr(tBu)-sulfamylbutyryl AM resin. Side-chain protection was employed as previously described for the synthesis of peptide α-thioesters by the Fmoc-protocol (J. A. Camarero et al., Protein Pept Lett 12, (2005)), except for the N-terminal Cys residue, which was introduced as Boc-Cys(Trt)-OH. After chain assembly, the alkylation, thiolytic cleavage and deprotection were performed as previously described (J. A. Camarero et al., Protein Pept Lett 12, (2005), J. Contreras et al., J. Control. Release 155 (2011)). Briefly, ≈100 mg of protected peptide resin was first alkylated two times with ICH₂CN (174 μL, 2.4 mmol; previously filtered through basic silica) and DIEA (82 μL, 0.46 mmol) in N-methylpyrrolidone (NMP) (2.2 mL) for 12 h. The resin was then washed with NMP (3×5 mL) and DCM (3×5 mL). The alkylated peptide resin was cleaved with HSCH₂CO₂Et (200 μL, 1.8 mmol) in the presence of a catalytic amount of sodium thiophenolate (NaSPh, 3 mg, 22 μmol) in dimethylformamide (DMF):DCM (3:4 v/v, 1.4 mL) for 24 h. The resin was then dried at reduced pressure. The side-chain protecting groups were removed by treating the dried resin with trifluoroacetic acid (TFA):H₂O:tri-isopropylsilane (TIS) (95:2:3 v/v, 5 mL) for 3-4 h at room temperature. The resin was filtered, and the linear peptide thioester was precipitated in cold Et₂O. The crude material was dissolved in the minimal amount of H₂O:MeCN (4:1) containing 0.1% TFA and characterized by HPLC and ES-MS. Cyclization and folding was accomplished by flash dilution of the corresponding MCo-52-2 linear α-thioester TFA crudes to a final concentration of ≈50 μM into freshly degassed 2 mM reduced glutathione (GSH), 50 mM sodium phosphate buffer at pH 7.5 for 18 h. Folded peptides were purified by semi-preparative HPLC using a linear gradient of 25-45% solvent B over 30 min. Pure peptides were characterized by HPLC and ES-MS FIG. 1 is a schematic of this “one-pot” synthetic method.

Construction of cyclotide expressing plasmids. Plasmids expressing the MCo-52-2 cyclotides were constructed using the pTXB1 expression plasmid (New England Biolabs), which contain an engineered Mxe Gyrase intein, respectively, and a chitin-binding domain (CBD) as previously described (Y. Ji et al., J. Am. Chem. Soc. 135, (2013), M. J. Campbell et al., Methods Mol. Biol. 2133 (2020)). Oligonucleotides coding for the different MCo-52-2 variants were synthesized, phosphorylated and PAGE purified by IDT DNA. Complementary strands were annealed in 20 mM sodium phosphate, 300 mM NaCl and the resulting double-stranded DNA (dsDNA) was purified using Qiagen's (Valencia, CA) miniprep column and buffer PN. pTXB1 plasmids was double digested with NdeI and SapI (NEB). The linearized vectors and the cyclotide encoding dsDNA fragments were ligated at 16° C. overnight using T4 DNA Ligase (New England Biolabs). The ligated plasmids were transformed into DH5a cells (Invitrogen) and plated on Luria Broth (LB)-agar containing ampicillin. Positive colonies were grown in 5 mL LB containing ampicillin at 37° C. overnight and the corresponding plasmids purified using a Miniprep Kit (Qiagen). Plasmids expressing the MCo-52-2 cyclotide precursors with an N-terminal TEV recognition sequence (Met-Glu-Asn-Leu-Tyr-Phe-Gln (SEQ ID NO: 6)) were cloned as follows. The DNA encoding TEV N-terminal recognition sequence was generated by PCR using the corresponding MCo-52-2-pTXB1 plasmid. The 5′ primer (5′-AAA CAT ATG GAA AAC CTG TAC TTC CAG TGC GGT TCT GGT TCT GG-3′ (SEQ ID NO: 7)) encoded an Nde I restriction site. The 3′ oligonucleotide (5′-GAT TGC CAT GCC GGT CAA GG-3′ (SEQ ID NO: 8)) introduced a Spe I restriction site during the PCR reaction. The PCR amplified product was purified, digested simultaneously with Nde I and Spe I and then ligated into an Nde I- and Spe I-treated plasmid pTXB-1 (New England Biolabs). The linearized vectors and the TEV-MCo-52-2 encoding dsDNA fragments were ligated at 16° C. overnight as described above. The ligated plasmids were transformed into DH5alpha cells and screened as described above. The DNA sequence of all the plasmids was confirmed by sequencing.

Expression and purification of recombinant MCo-52-2 cyclotides. BL21(DE3) (Novagen) were transformed with MCo-52-2 encoding plasmids (see above). Expression was carried out in LB medium (1 L) containing ampicillin (100 μg/mL) at 30° C. for 4 h respectively. Briefly, 5 mL of an overnight starter culture derived from either a single clone or single plate were used to inoculate 1 L of LB media. Cells were grown to an OD at 600 nm of ≈0.6 at 37° C., and expression was induced by the addition of isopropyl-β-D-thiogalactopyranoside (IPTG) to a final concentration of 0.3 mM at 30° C. for 4 h. The cells were then harvested by centrifugation. For fusion protein purification, the cells were resuspended in 30 mL of lysis buffer (0.1 mM EDTA, 1 mM PMSF, 50 mM sodium phosphate, 250 mM NaCl buffer at pH 7.2 containing 5% glycerol) and lysed by sonication. The lysate was clarified by centrifugation at 15,000 rpm in a Sorval SS-34 rotor for 30 min. The clarified supernatant was incubated with chitin-beads (2 mL beads/L cells) (New England Biolabs), previously equilibrated with column buffer (0.1 mM EDTA, 50 mM sodium phosphate, 250 mM NaCl buffer at pH 7.2) at 4° C. for 1 h with gentle rocking. The beads were extensively washed with 50 bead-volumes of column buffer containing 0.1% Triton X100 and then rinsed and equilibrated with 50 bead-volumes of column buffer. For the purification of TEV-MCo-52-2-intein-CBD fusion proteins, the beads were washed with 50 bead-volumes of TEV reaction buffer (50 mM Tris·HCl, 0.5 mM EDTA pH 8.0). Proteolytic cleavage of the TEV sequence was performed on the column by complementing the buffer with 3 mM reduced GSH and adding TEV protease to a final concentration of 0.1 mg/mL. The proteolytic reaction was kept at 4° C. overnight with gentle rocking. Once the proteolytic step was completed, the column was then washed with 50-bead volumes of column buffer. Chitin beads containing the different purified MCo-52-2-Intein-CBD fusion proteins were cleaved with 50 mM GSH in degassed column buffer. The cleavage reactions were kept for up to 1-2 days at 25° C. with gentle rocking. Once the cleavage reaction was complete, the supernatant of the cleavage reaction was separated by filtration and the beads were washed with additional column buffer to reach a final concentration of 5 mM GSH, and the folding was allow to proceed with gently rocking at 4° C. for 48 h. Folded MCo-52-2 cyclotides were purified by semipreparative HPLC using a linear gradient of 25-45% solvent B over 30 min. Purified MCo-52-2 cyclotides were characterized by C18-RP-HPLC and ES-MS; and quantified by UV-vis spectroscopy.

Production of cyclotide MCo-52-2-cTAT. The scheme used to produce cyclotide MCo-52-2-cTAT is depicted in FIG. 2. Briefly, conjugation of N₃-EG₃-CONH-cTAT peptide was done through the loop 2 of cyclotide MCo-52-2-Alk, where residue Ser15 was replaced by a Lys residue where the side-chain e-amino group was acylated with 5-pentynoic acid (FIG. 2). Conjugation of the azido-labeled cTAT peptide was performed using a Huisgen copper(I)-catalyzed azide-alkyne 1,3-dipolar cycloaddition (CuAAC) (J. Contreras et al., J. Control. Release 155 (2011), J. A. Camarero et al., Biopolymers 90, (2008)). Chemical synthesis of cyclotides facilitates the introduction of non-natural amino acids containing the alkyne group that can be later bioconjugated to azide-containing fluorescent dyes using a CuAAC. Alternatively, alkyne-modified cyclotides can also be recombinantly expressed as recently described (K. Jagadish et al., Angew Chem Int Ed Engl 52, (2013)).

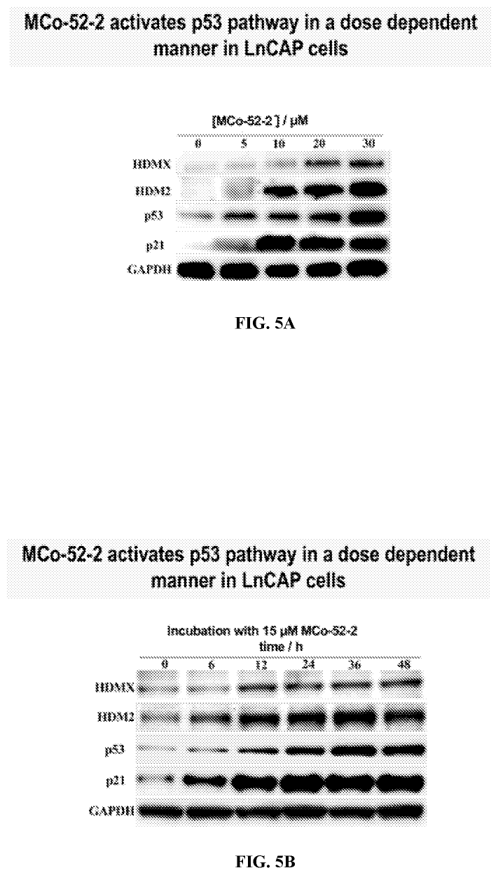

In vitro inhibition of Hdm2-HdmX complex competition experiments. In vitro IC₅₀ values were measured by inhibition competition experiments using the FRET-based reporter formed by YPet-Hdm2 and CyPet-HdmX. Ypet-Hdm2 and CyPet-HdmX were produced as previously described (Y. Ji et al., J. Am. Chem. Soc. Soc. 135, (2013)). Briefly, a solution of YPet-Hdm2 (200 nM) and CyPet-HdmX (500 nM) 10 mM phosphate buffer, 150 mM NaCl buffer at pH 7.2 was titrated with increasing amounts of inhibitor (ranging from 0 to 10 μM). The decrease in fluorescence signal at 525 nm (excited at 414 nm) was measured and plotted against the concentration of free inhibitor. The resulting plot was fitted to a single binding site competition curve using the Prism (GraphPad) software package. See FIGS. 4 to 6.

Cell viability assay in several human cancer cell lines (See Tables 1 to 10). Cell lines were cultured in RPMI 1640 (or recommended medium when indicated) medium supplemented with 10% fetal calf serum, penicillin (50 IU/mL) and streptomycin (50 g/mL) at 37° C. in 5% CO₂. Cell viability studies was carried using the MTT (3-(4,5-dimethylthiazol-2-yl)-2,5-diphenyltetrazolium bromide) assay as previously described (31). Briefly, ≈2×10³ CRC cells were be seeded in 96-well microtiter plates in 100 μL RPMI 1640 in the presence of 10% calf serum. After 24 h incubation the cells were washed with PBS and treated with 30 μL/well of PBS or RPMI 1640 (or recommended) media containing cyclotides MCo-52, MCoTI-I (used as negative control), nutlin-3a (used as positive control) or MI-77301 (used as positive control for p53-mutated or p53-null cells) at the indicated doses for 1 h at 37° C. in 5% CO₂. After 1 h, 210 μL/well of full complemented media was added, the cells were grown for 48 h and then treated with 20 μL of a solution of MTT (5 mg/mL) for 2 h. The medium was discarded and DMSO (100 L/well) was added to each well and incubated with gentle shaking for 20 min at room temperature. The absorbance at 595 nm of the solution is analyzed using a Tecan Genios Multifunctional Microplate Reader (Tecan System Inc) and the background at 670 nm subtracted. RNA-Seq analysis was performed using state-of-the-art bioinformatics tools. Briefly, raw RNA-Seq reads were mapped to human genome (hg38) using STAR aligner (v2.7.3a). Gencode Release 36 reference was used for annotation. Transcript and gene expression levels were quantified using the IsoEM (37) with default parameters. Counts per million (CPM) method is used for normalization. Differential expression was done using DESeq2 with default parameters. Genes involved in different biological pathways (human) were searched from Geneontology (http://geneontology.org/). Bioinformatics results can be validated by using Partek Flow (v10.0.21.0912), a bioinformatics pipeline developed by Zymo Research (https://www.zymoresearch.com/).

In vivo efficacy and safety of MCo-52-2-c-TAT cyclotide in cell derived xenograft (CDX) models. MCo-52-2-cTAT cyclotide was tested for in vivo efficacy against several CRC CDX models using SW480, HCT116 (null p53), and HT29 cell lines. Luciferase labeled reporter cell lines was generated. 1×10⁶ cells were suspended in 50 μL 1×PBS and 50 μL matrigel (1:1 ratio) and injected subcutaneously in the flanks of the 5-weeks old, inbred nude (Nu/J) male mice. Cells before implantation and mice immediately after the injections undergo imaging using IVIS 100 Bioluminescence/optical imaging system (Xenogen Corporation, CA, USA). Once tumors are palpable (˜50-100 mm³), mice were treated with vehicle (5% dextrose in saline), cyclotide MCo-52-2-cTAT (25 mg/kg, IP, 3 times a week) or MI-77301 (30 mg/kg, P.O., once daily) as a positive control (38). Ten mice per treatment group are believed to be necessary to study tumor growth and survival. Initial drug doses are selected based on preliminary data for MCo-52-2-cTAT which has been given safely to mice. Mice are treated with cyclotides (doses mentioned above) for 14 days or a maximum of 32 days based on tumor growth/endpoint in the control group. Mice were monitored daily for any sign of distress and toxicity due to treatments. Body weight and tumor volume were measured three times a week. Survival curves were calculated for treated and untreated murine cohorts to estimate efficacy of MCo-52-2 cyclotides on the survival, and tumor burden. The primary endpoint were either tumor volume at the time of euthanizing or the “area under the curve” (AUC) for the size of the tumor over time.

Results of the in vitro cell assays are provided in Tables 1 to 10. MCo-52-2 is abbreviated as 52-2 MCo-52-2-cTAT is abbreviated as 52-2-cTAT. MCoTI-I and cTAT are used as controls to show that toxicity is not related to cTAT and/or the cyclotide wild-type sequence MCoTI-I. Nutlin 3a is used as positive control in cells with wild-type-53 phenotype. https://www.medchemexpress.com/Nutlin-3a.html?src=google-product&gclid=CjOKCQjwma6TBhDIARIsAOKuANyQFfg3GUDGu-IiN8EeKyOPaOC8qB6qB-_Y_pLqNOn6jmPHN8Jlu-YaAq9rEALw_wcB. MI77301 is used as positive control in cells with null- or mutated-53 phenotypes. https://www.medchemexpress.com/SAR405838.html.

Tables 1 through 10 show in vitro activity of cyclotide MCo-52-2-cTAT against different human cancer cell lines, some with null-, mutated, or wildtype p53 phenotypes. Cell viability was evaluated using the MTT assay as described above.

TABLE 1
Human Colorectal Cancer Cells

| | | P53 | | EC₅₀ / μM | | | | | |
|---|---|---|---|---|---|---|---|---|---|
| Cell line | Type of Cancer | status | Type of mutation | 52-2 | 52-2-cTAT | Nutlin 3A | MCoTI-I | cTAT | MI-77301 |
| HCT116^{+/+} | Colon carcinoma | WT | | >100 | 10.9 ± 1.3 | 6.0 ± 0.8 | >100 | >100 | 2.7 ± 0.2 |
| | Primary tumor | | | | | | | | |
| HCT116^{-/-} | Colon carcinoma | Null | | >100 | 24.4 ± 2.3 | >100 | >100 | >100 | 23.7 ± 1.9 |
| LoVo | Colon adenocarcinoma | WT | | >100 | 10.7 ± 0.9 | 1.5 ± 0.1 | >100 | >100 | ND |
| | metastatic site: Left | | | | | | | | |
| | supraclavicular lymph node | | | | | | | | |
| DLD-1 | Colon adenocarcinoma | Mutant | Ser241Phe | >100 | 20.7 ± 2.2 | >100 | >100 | >100 | ND |
| | Primary | | | | | | | | |
| SW620 | Colon adenocarcinoma | Mutant | Pro309Ser Hertozygous | >100 | 15.3 ± 2.0 | >100 | >100 | >100 | ND |
| | metastatic site: Lymph node | | | | | | | | |
| HT29 | Colon adenocarcinoma | Mutant | Arg273His | >100 | 9.9 ± 0.9 | >100 | >100 | >100 | ND |
| | Primary tumor | | Hertozygous | | | | | | |
| SW626 | Colon adenocarcinoma | Mutant | | >100 | 28.2 ± 4.6 | >100 | >100 | >100 | ND |
| | metastatic site: Ovary | | | | | | | | |
| Caco-2 | Colon adenocarcinoma | Mutant | p. Glu204Ter (c. 610 > T) | >100 | 11.7 ± 0.8 | >50 | >100 | >100 | >50 |
| | | | Homozygous | | | | | | |
| SW480 | Colon adenocarcinoma\ | Mutant | p. Pro309Ser | >100 | 10.9 ± 1.3 | 58.7 ± 7.5 | >100 | >100 | 26.1 ± 3.3 |
| | Primary tutor | | (c. 925° C. > T) | | | | | | |
| | | | Hertozygous | | | | | | |

TABLE 2
Mutation Status of Various Human Cancer Cell Lines

| | | | | | EC₅₀ / μM | | |
|---|---|---|---|---|---|---|---|
| | Cell line | Type of Cancer | P53 status | Type of mutation | MCO-52-2 | Nutlin 3A | MI-77301 |
| Lung | NCl-H23 | Lung adenocarcinoma Primary | Mutant | p. Met246lle (c. 738G > C) | 8.8 ± 0.9 | >50 | 10.7 ± 1.9 |
| carcinoma | | | | Homozygous | | | |
| | NCl-H522 | Lung adenocarcinoma Primary | Mutant | p. Pro191fs*56 | 24.6 ± 2.5 | >50 | 22.7 ± 2.8 |
| | | | (Deletion) | (c. 571delC) | | | |
| | | | | Homozygous | | | |
| | NCl-H1299 | Lung large cell carcinoma | Null | c. (del) | 9.1 ± 0.8 | >100 | 21.5 ± 2.5 |
| | | Metastatic site: Lymph node | | Homozygous | | | |
| | NCl-H69 | Lung small cell carcinoma | Mutant | p. Glu171Ter (c. 511G > T) | 4.4 ± 0.6 | >50 | 31.7 ± 5.2 |
| | | Metastatic site: Pleural effusion | | Homozygous | | | |
| | NCl-H526 | Lung small cell carcinoma | Null | c. 97-1G > C Homozygous | 4.7 ± 0.5 | >100 | 9.7 ± 1.8 |
| | | Metastatic site: Bone marrow | | | | | |
| Colorectal | Caco-2 | Colon adenocarcinoma | Mutant | p. Glu204Ter (c. 610 > T) | 11.7 ± 0.8 | >50 | >50 |
| carcinoma | | | | Homozygous | | | |
| | SW480 | Colon adenocarcinoma\ | Mutant | p. Pro309Ser (c. 925C > T) | 10.9 ± 1.3 | 58.7 ± 7.5 | 26.1 ± 3.3 |
| | | Primary tutor | | Hertozygous | | | |

TABLE 3
Mco-52-2-cTAT shows activity in CRC cells independently of the microsatellite instability (MSI) state

| | | P53 | MSI | | EC₅₀ / μM |
|---|---|---|---|---|---|
| Cell line | Type of Cancer | status | status | Type of mutation | MCo-52-2-cTAT |
| HCT116^{+/+} | Colon carcinoma | WT | MSI | | 10.9 ± 1.3 |
| | Primary tumor | | | | |
| HCT116^{-/-} | Colon carcinoma | Null | MSI | | 14.4 ± 2.3 |
| LoVo | Colon adenocarcinoma metastatic site: | WT | MSI | | 10.7 ± 0.9 |
| | Left supraclavicular lymph node | | | | |
| SW620 | Colon adenocarcinoma metastatic site: | Mutant | MSS | Pro309Ser | 15.3 ± 2.0 |
| | Lymph node | | | Hertozygous | |
| HT29 | Colon adenocarcinoma | Mutant | MSS | Arg273His | 9.9 ± 0.9 |
| | Primary tumor | | | Hertozygous | |
| Caco-2 | Colon adenocarcinoma | Mutant | MSS | p. Glu204Ter | 11.7 ± 0.8 |
| | | | | (c. 610 > T) | |
| | | | | Homozygous | |
| SW480 | Colon adenocarcinoma\ | Mutant | MSS | p. Pro309Ser | 10.9 ± 1.3 |
| | Primary tutor | | | (c. 925C > T) | |
| | | | | Hertozygous | |

TABLE 4
Murine Cancer Cells

| | | p53 | Type of | EC₅₀ / μM | | | | | |
|---|---|---|---|---|---|---|---|---|---|
| Cell line | Type of Cancer | Status | mutation | 52-2 | 52-2-cTAT | Nutlin 3A | MCoTI-I | cTAT | MI-77301 |
| MSCLC | Murine Small Cell lung | ND | ND | >100 | 28.9 ± 3.6 | >100 | >100 | >100 | 15.8 ± 2.2 |
| 494 | cancer | | | | | | | | |
| 4T1 | Murine Breast Cancer | WT | N/A | >100 | 8.9 ± 1.6 | 28.5 ± 8.6 | >100 | >100 | ND |
| CT26 | Murine Colon Cancer | WT | N/A | >100 | 4.9 ± 0.7 | 9.8 ± 1.2 | >100 | >100 | ND |
| LLC-1 | Murine Lung Cancer | WT | N/A | >100 | 15.1 ± 3.3 | 9.4 ± 1.2 | >100 | >100 | ND |

TABLE 5
Human Leukemia Cancer Cells

| | | p53 | | EC₅₀ / μM | | | | | |
|---|---|---|---|---|---|---|---|---|---|
| Cell line | Type of Cancer | Status | Type of mutation | 52-2 | 52-2-cTAT | Nutlin 3A | MCoTI-I | cTAT | MI-77301 |
| HL-60 | Adult acute myeloid | Null | Homozygous | >100 | 4.1 ± 0.5 | 33.3 ± 4.4 | >100 | >100 | ND |
| | leukemia | | | | | | | | |
| Jurkat | Childhood T acute | Mutant | p. Arg196Ter | >100 | 4.4 ± 0.4 | 45.2 ± 2.4 | >100 | >100 | 12.1 ± 1.6 |
| | lympoblastic leukemia | | (c. 586C > T) | | | | | | |
| | | | Hertozygous | | | | | | |
| U937 | Adult acute monocytic | Mutant | (p. Val173Trpfs*59) | >100 | 4.4 ± 0.6 | >50 | >100 | >100 | 9.6 ± 2.2 |
| | leukemia | (Addition/ | c. 559 + 1G > A | | | | | | |
| | | FShift) | Homozygous | | | | | | |
| MOLM-13 | Adult acute myeloid | WT | | >100 | 1.1 ± 0.1 | 1.4 ± 0.1 | >100 | >100 | 0.7 ± 0.1 |
| | leukemia | | | | | | | | |
| MV4-11 | Childhood acute | WT | | >100 | 4.6 ± 0.7 | 0.7 ± 0.1 | >100 | >100 | 0.3 ± 0.03 |
| | monocytic leukemia | | | | | | | | |

TABLE 6
Human Lung Cancer Cells

| | | p53 | | EC₅₀ / μM | | | | | |
|---|---|---|---|---|---|---|---|---|---|
| Cell line | Type of Cancer | Status | Type of mutation | 52-2 | 52-2-cTAT | Nutlin 3A | MCoTI-I | cTAT | MI-77301 |
| NCl-A549 | Lung adenocarcinoma | WT | | >100 | 12.2 ± 1.8 | 6.6 ± 0.7 | >100 | >100 | 15.8 ± 2.2 |
| NCl-H1944 | Lung adenocarcinoma | WT | | >100 | 9.5 ± 1.5 | 22.1 + 4.7 | >100 | >100 | ND |
| NCl-H292 | Lung mucoepidermoid | WT | | 61.8 ± 15.8 | 8.4 ± 0.1 | 19.2 ± 2.9 | >100 | >100 | ND |
| | carcinoma | | | | | | | | |
| NCl-H1395 | Lung adenocarcinoma | WT | | >100 | 4.6 ± 0.6 | 23.1 ± 5.6 | >100 | >100 | ND |
| | epithelial cell | | | | | | | | |
| NCl-H2347 | Lung adenocarcinoma | Mutant | c. 375G > A; | >100 | 8.6 ± 1.0 | 25.3 ± 5.5 | >100 | >100 | ND |
| | Non-small cell lung | | Thr125Thr | | | | | | |
| | cancer | | Impairs p53 | | | | | | |
| | | | splicing | | | | | | |
| | | | dramatically | | | | | | |
| NCl-H1975 | Lung adenocarcinoma | Mutant | p. Arg273His | >100 | 12.3 ± 1.2 | >100 | >100 | >100 | ND |
| | Non-small cell lung | | homozygous | | | | | | |
| | cancer, Primary | | | | | | | | |
| NCl-H23 | Lung adenocarcinoma | Mutant | p. Met246ile | >100 | 8.8 ± 0.9 | >50 | >100 | >100 | 10.7 ± 1.9 |
| | Primary | | (c. 738G > C) | | | | | | |
| | | | Homozygous | | | | | | |
| NCl-H522 | Lung adenocarcinoma | Mutant | p. Pro191fs*56 | >100 | 24.6 ± 2.5 | >50 | >100 | >100 | 22.7 ± 2.8 |
| | Primary | (Deletion) | (c. 571delC) | | | | | | |
| | | | Homozygous | | | | | | |
| NCl-H1299 | Lung large cell carcinoma | NULL | c. (del) | >100 | 9.1 ± 0.8 | >100 | >100 | >100 | 21.5 ± 2.5 |
| | Metastic site: Lympth | | Homozygous | | | | | | |
| | node | | | | | | | | |
| NCl-H69 | Lung small cell carcinoma | Mutant | p. Glu171Ter | >100 | 4.4 ± 0.6 | >50 | >100 | >100 | 31.7 ± 5.2 |
| | Metastic site: Pleural | | (c. 511G > T) | | | | | | |
| | effusion | | Homozygous | | | | | | |
| NCl-H526 | Lung small cell carcinoma | Null | c. 97-1G > C | >100 | 4.7 ± 0.5 | >100 | >100 | >100 | 9.7 ± 1.8 |
| | Metastic site: Bone | | Homozygous | | | | | | |
| | marrow | | | | | | | | |

TABLE 7
Cell Viability Assay of 52-2 on Ovarian cells

| Cell line | 52-2 | 52-2-cTAT | Nutlin 3A | | MCoTI-I | cTAT | p53 Status |
|---|---|---|---|---|---|---|---|
| A2780 | >100 μM | 9.7 ± 0.5 μM | 9.3 ± 0.5 | μM | >100 μM | >100 μM | WT |
| SW626 | >100 μM | 27.5 ± 0.7 μM | 50.3 ± 0.2 | μM | >100 μM | >100 μM | Mutant |
| NIHOVCAR3 | >100 μM | 7.8 ± 0.1 μM | 57.7 ± 0.1 | μM | >100 μM | >100 μM | Mutant |
| SKOV 3 | >100 μM | 19.1 ± 0.1 μM | >100 | μM | >100 μM | >100 μM | Mutant |
| ES-2 | >100 μM | 6.38 ± 0.1 μM | 92.3 ± 1.2 | μM | >100 μM | >100 μM | Mutant |

TABLE 8

| | | p53 | | EC₅₀ / μM | | | | | |
|---|---|---|---|---|---|---|---|---|---|
| Cell line | Type of Cancer | Status | Type of mutation | 52-2 | 52-2-cTAT | Nutlin 3A | MCoTI-I | cTAT | MI-77301 |
| Capan-1 | Pancreatic ductal | Mutant | Ala159Val | >100 | 3.3 ± 0.3 | >100 | >100 | >100 | ND |
| | adenocarcinoma | | Homozygous | | | | | | |
| | metastatic site: Liver | | | | | | | | |
| AsPC-1 | Pancreatic ductal | Mutant | Cys135Alafs*35 | >100 | 11.5 ± 1.5 | >100 | >100 | >100 | ND |
| | adenocarcinoma | | Heterozygous | | | | | | |
| | metastatic site: Ascites | | | | | | | | |
| SW1990 | Pancreatic | Mutant | Pro191del | >100 | 11.9 ± 1.3 | >100 | >100 | >100 | ND |
| | adenocarcinoma | (Deletion) | Heterozygous | | | | | | |
| | metastatic site: spleen | | | | | | | | |
| BxPC-3 | Pancreatic ductal | Mutant | p. Tyr220Cys | >100 | 10.6 ± 1.0 | 38.5 ± 5.1 | >100 | >100 | 17.3 ± 2.3 |
| | adenocarcinoma | | homozygous | | | | | | |
| | Primary | | | | | | | | |
| MIA PaCa-2 | Pancreatic ductal | Mutant | p. Arg248Trp | >100 | 15.4 ± 1.6 | >50 | >100 | >100 | 10.5 ± 1.6 |
| | adenocarcinoma | | Homozygous | | | | | | |
| | Primary | | | | | | | | |
| PANC-1 | Pancreatic ductal | Mutant | p. Arg273His | >100 | 20.5 ± 2.5 | 41.9 ± 6.9 | >100 | >100 | 12.7 ± 1.6 |
| | adenocarcinoma | | Homozygous | | | | | | |
| | Primary tumor | | | | | | | | |
| HPAF-II | Pancreatic ductal | Mutant | p. Pro151Ser | >100 | 14.6 ± 1.8 | >50 | >100 | >100 | 6.7 ± 1.0 |
| | adenocarcinoma | | Homozygous | | | | | | |
| | metastatic site: Ascites | | | | | | | | |
| CFPAC-1 | Pancreatic ductal | Mutant | p. Cys242Arg | >100 | 13.4 ± 1.8 | >50 | >100 | >100 | 22.1 ± 1.6 |
| | adenocarcinoma | | Homozygous | | | | | | |
| | metastatic site: Liver | | | | | | | | |

TABLE 9
Human Pancreatic Cancer Cells

| | | p53 | | EC₅₀ / μM | | | | | |
|---|---|---|---|---|---|---|---|---|---|
| Cell line | Type of Cancer | Status | Type of mutation | 52-2 | 52-2-cTAT | Nutlin 3A | MCoTI-I | cTAT | MI-77301 |
| Capan-1 | Pancreatic ductal | Mutant | Ala159Val | >100 | 3.3 ± 0.3 | >100 | >100 | >100 | ND |
| | adenocarcinoma | | Homozygous | | | | | | |
| | metastatic site: Liver | | | | | | | | |
| AsPC-1 | Pancreatic ductal | Mutant | Cys135Alafs*35 | >100 | 11.5 ± 1.5 | >100 | >100 | >100 | ND |
| | adenocarcinoma | | Heterozygous | | | | | | |
| | metastatic site: Ascites | | | | | | | | |
| SW1990 | Pancreatic | Mutant | Pro191del | >100 | 11.9 ± 1.3 | >100 | >100 | >100 | ND |
| | adenocarcinoma | (Deletion) | Heterozygous | | | | | | |
| | metastatic site: spleen | | | | | | | | |
| BxPC-3 | Pancreatic ductal | Mutant | p. Tyr220Cys | >100 | 10.6 ± 1.0 | 38.5 ± 5.1 | >100 | >100 | 17.3 ± 2.3 |
| | adenocarcinoma | | homozygous | | | | | | |
| | Primary | | | | | | | | |
| MIA PaCa-2 | Pancreatic ductal | Mutant | p. Arg248Trp | >100 | 15.4 ± 1.6 | >50 | >100 | >100 | 10.5 ± 1.6 |
| | adenocarcinoma | | Homozygous | | | | | | |
| | Primary | | | | | | | | |
| PANC-1 | Pancreatic ductal | Mutant | p. Arg273His | >100 | 20.5 ± 2.5 | 41.9 ± 6.9 | >100 | >100 | 12.7 ± 1.6 |
| | adenocarcinoma | | Homozygous | | | | | | |
| | Primary tumor | | | | | | | | |
| HPAF-II | Pancreatic ductal | Mutant | p. Pro151Ser | >100 | 14.6 ± 1.8 | >50 | >100 | >100 | 6.7 ± 1.0 |
| | adenocarcinoma | | Homozygous | | | | | | |
| | metastatic site: Ascites | | | | | | | | |
| CFPAC-1 | Pancreatic ductal | Mutant | p. Cys242Arg | >100 | 13.4 ± 1.8 | >50 | >100 | >100 | 22.1 ± 1.6 |
| | adenocarcinoma | | Homozygous | | | | | | |
| | metastatic site: Liver | | | | | | | | |

TABLE 10
Human Prostate Cancer Cells

| | | | | All values are in μM | | | | |
|---|---|---|---|---|---|---|---|---|
| Cell line | Type of Cancer | p53 Status | Type of Mutation | 52.2 | 52-2-cTAT | Nutlin 3A | MCoTI-I | cTAT |
| LNCaP | Prostate carcinoma | WT | | >100 | 6.3 ± 1.1 | 19.7 ± 1.3 | >100 | >100 |
| DU145 | Prostate carcinoma | Mutant | p. Val274Phe, | >100 | 37.0 ± 1.2 | >100 | >100 | >100 |
| | metastatic site: Brain | | p. Pro223Leu | | | | | |
| | | | heterozygous | | | | | |
| PC-3 | Prostate carcinoma | Mutant | p. Lys139Argfs*31 | >100 | 23.2 ± 0.6 | >100 | >100 | >100 |
| | metastatic site: Bone | | homozygous | | | | | |
| VCaP | Prostate carcinoma | Mutant | p. Arg248Trp | | | | | |
| | metastatic site: Bone; | | (c. 742C > T) | | | | | |
| | vertebra | | | | | | | |
| DuCaP | Prostate carcinoma | Mutant | p. Arg248Trp | | | | | |
| | metastatic site: Brain | | (c. 742C > T) | | | | | |
| | dura mater. | | | | | | | |

Mice xenografts studies. HCT116 p53^{-/-} and CT26 xenografts were established by injecting 100 uL suspension of basal RPMI containing 0.5×10⁶ cells into the rear right flanks of female nude mice (nu/nu) mice (HCT116) or Balb/C (CT26). When tumors reached an average volume of ≈100 mm³, cohorts (n≥4) were treated with vehicle (5% dextrose in water, D5W), MCo-52-2-cTAT (40 mg/kg), or Nutlin-3 (20 mg/kg, 17.2 mmol/kg) once daily for up to 38 days by intravenous injection (MCo-52-2-cTAT and vehicle) or by intraperitoneal administration (Nutlin-3) for HCT116 xenografts; or with vehicle (D5W), MCo-52-2-cTAT (25 mg/kg) three times per week by intraperitoneal injection. Compounds were prepared in a D5W at a final volume of 50 μL (peptide) or 100 μL (Nutlin-3). Health checks were performed daily to observe parameters such as body conditioning score, overall appearance and cleanliness, strength of grip, skin color and tone, mobility, gait and activity level as indicators of potential drug related toxicities. Individual weights were recorded thrice weekly, comparing the control and treatment groups as an additional indicator of tolerance of drug treatment as well as providing the average weight for calculation of drug dosing. Tumor size was measured with calipers. Tumor volume was calculated by measuring tumor size in two dimensions and applying those measurements to the calculation V=d²×D/2, where d and D equal to the smaller and larger of the two measurements, respectively. Mice bearing tumors larger than 2.4 cm³ were removed from the study, sacrificed and necropsies performed to gather tumor and organ samples for histological analysis. Tumor and tissue samples were perfused with PBS, after which portions were either fixed in 10% formalin overnight then transferred to 100% EtOH or snap-frozen in liquid nitrogen and stored at −70° C. until analysis.

Quantitative RT-PCR. HCT116 p53^{+/+} and CT26 subcutaneous tumors were excised, flash frozen and the RNA extracted using the RNeasy Mini kit (QIAGEN). Total RNA was reverse transcribed to cDNA using M-MLV reverse transcriptase (Promega). The generated cDNA was amplified with power SYBR Green PCR master mix (Applied Biosystems) on a 96 well plate and measured the relative transcript levels by qRT-PCR on an ABI 7900HT Fast Real-Time PCR System (Applied Biosystems). Specific primers for HDM2, HDMX, p21 and the GAPDH control were used. The amplification reactions were done in triplicate in 96-well optical plates. Threshold-cycle (Ct) values were automatically calculated for each replicate and used to determine the relative expression of the gene of interest relative to β-actin.

Immunohistochemistry. Formalin-fixed, paraffin-embedded (FFPE) tumor samples were sectioned at a thickness of 5 μm and mounted on pre-cleaned, charged glass slides. For immunohistochemisty, tumor sections were de-paraffinized in 3×5 min changes of clear-rite 3 (Microm International). Samples were then rehydrated by treatment with 100% ethanol (EtOH) (2×10 min) followed by treatment with 95% EtOH in H₂O (2×10 min) and then washed with pure H₂O. For antigen unmasking, slides were incubated at 95° C. in 10 mM sodium citrate buffer containing 0.05% tween-20 at pH 6.0 for 10 min. The slides were cooled to room temperature and washed 3×2 min in phosphate-buffered saline without calcium and magnesium salts (PBS) (3×2 min). Immunohistochemical staining was carried out using the Ultravision ONE Detection System (Thermo Scientific) containing horseradish-peroxidase polymer (Thermo Scientific) and DAB Plus Chromagen (Thermo Scientific) according to the manufacturer's directions. Briefly, tissues were incubated with hydrogen peroxide blocking (Thermo Scientific) reagent to quench endogenous peroxides, and then washed with PBS (4×2 min). Ultra V blocking agent (Thermo Scientific) was applied to prevent non-specific binding. The corresponding primary antibodies were prepared at a concentration of 1 μg/mL in PBS containing 1.5% normal goat serum. Following overnight incubation at 4° C. in the presence of antibody, tissue sections were washed as before and incubated with HRP polymer prior to application of DAB chromagen/substrate. Tissue sections were incubated with DAB for 3 min to allow color to develop. Slides were washed with pure H₂O (4×5 min), counterstained with hematoxylin solution (Microm International) for 1 min and immediately washed with pure H₂O. Slides were dehydrated with successive washes of 95% and 100% EtOH, and clearite-3, then mounted with clarion mouting media (Sigma-Aldrich) and glass coverslips. Slides were air-dried overnight and visualized using Ziess Axioskop light microscope equipped with 5× and 20× objectives and motic digital camera.

### Discussion

Cyclotide MCo-52-2-cTAT efficiently antagonizes the RING-mediated dimerization of Hdm2/HdmX inhibiting its E3 ligase activity. Applicant developed a cell-based screening of genetically-encoded libraries of cyclotides for Hdm2/HdmX antagonists that has allowed the selection of a bioactive cyclotide, MCo-52-2-cTAT. This cyclotide that was able to bind the RING domains of both HdmX and Hdm2 (with K_{d} values ranging from 20 to 80 nM) and inhibit the formation of the RING-mediated Hdm2/HdmX complex (IC₅₀≈40 nM). As expected, cyclotide MCo-52-2 was also able to specifically inhibit the E3 ligase activity of Hdm2 in vitro in a dose-dependent fashion using a commercial kit (FIG. 4B). This cyclotide can be efficiently produced by chemical synthesis using solid-phase peptide synthesis (SPPS) or heterologous expression systems (28). Heterologous expression of cyclotides allows introduction of NMR-active isotopes, such as ¹³C and ¹⁵N to study the structure of this cyclotide and how interacts with the RING-domains of Hdm2 and HdmX. Using this approach, Applicant solved the structure of cyclotide MCo-52-2 complexed to the RING domain of HdmX in solution (FIG. 4A). The structure of the complex shows that residues located at loops 1 and 5 are strongly interacting with the C-terminal portion of the RING domain, which is critical for the RING-induced dimerization required for the E3 ligase activity of Hdm2/HdmX (FIG. 4A).

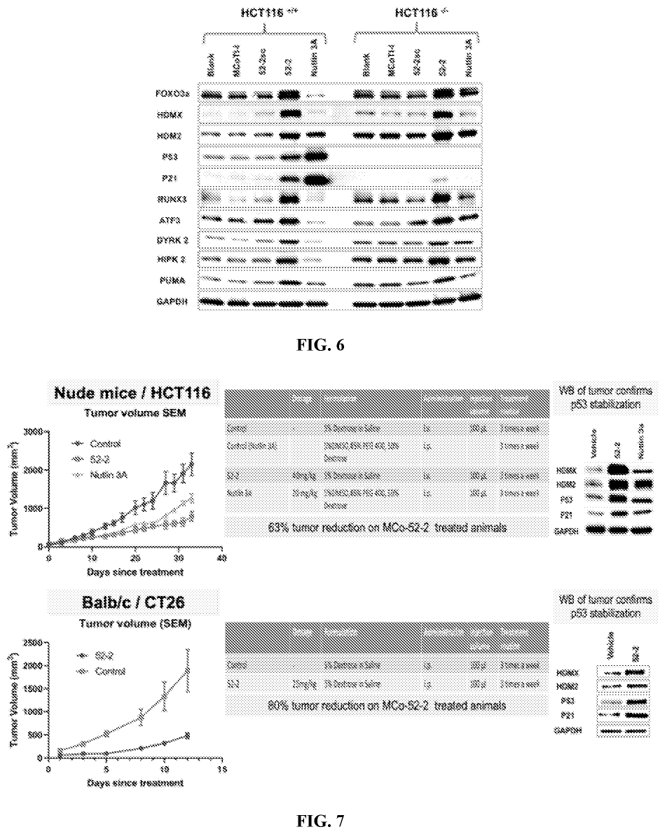

Cyclotide MCo-52-2-cTAT shows activity both in vitro and in vivo. Cyclotide MCo-52-2-cTAT was able to activate the p53 pathway in wild type-p53 cancer cells (LNCaP) in a dose-dependent fashion (FIG. 5A). Stabilization of p53 could be observed just 8 h after treating the cells with the bioactive cyclotide (FIG. 5B), likely indicating the cyclotide crosses the cellular membrane into the cytosol through an endocytic mechanism as previously described for this type of microproteins (30, 31). As expected, treatment of cancer cells with cyclotide MCo-52-2-cTAT was also able to stabilize ATF3, FOXO3a, RUNX3, DYRK2 and HIPK2, in wild-type HCT116 and HCT116^{p53}^{_{−}}^{/}^{_{−}} cell lines (FIG. 6). These transcription factors (ATF3, FOXO3a and RUNX3) and kinases (DYRK2 and HIPK2) have been described to be regulated by the E3 ligase activity of Hdm2/HdmX (6). Preliminary RNA-Seq analysis of HCT116 cancer cells treated with cyclotide MCo-52-2 clearly shows that inhibition of Hdm2/HdmX E3 ligase affects the mRNA level of many genes involved in the regulation of immune response, cell cycle, and apoptosis. As shown in Tables 1 to 10 cyclotide MCo-52-2 also showed significant activity against several human cancer cell lines including several human CRC cell lines with wild-type p53 phenotypes, and several other cancer cell lines with mutant- and null-p53 phenotypes. More importantly, this bioactive cyclotide significantly reduced tumor growth in two murine models of colorectal carcinoma using intravenous (HCT116-nude mice) and intraperitoneal (CT26-Balb/c mice) dosing (FIG. 7). In both cases the peptide was able to inhibit tumor growth by 60% (HCT116-nude mice) and 80% (CT26-Balb/c mice) (FIG. 7). The superior activity of cyclotide MCo-52-2 observed using the immunocompetent syngeneic animal model could suggest that genes involved on the anti-tumoral immune response may be stabilized/activated by inhibition of the Hdm2/HdmX E3 ligase activity. It is also interesting that Balb/C mice treated with cyclotide MCo-52-2 by intraperitoneal injection at 25 mg/kg, 3 times per week for 28 days did not show any significant IgG titer against the cyclotide MCo-52-2 when compared to that of untreated animals, again emphasizing the poor immunogenicity of this type of microproteins under the conditions used in this preliminary study. Altogether, these results highlight the therapeutic potential of MCoTI-based cyclotides and the power of molecular evolution to screen and select potent cyclotides against specific protein-protein interactions.

To Applicant's knowledge the disclosed compounds are the first reported inhibitors for Hdm2/HdmX that specifically targets its E3 ligase activity and exhibits good anticancer properties both in vitro and in vivo. These compounds represent a new type of inhibitor that stabilizes p53 not by antagonizing the p53-binding domains of Hdm2/HdmX such as nutlin-like compounds do (e.g., nutlin 3a, MI-77301, among others), but by inactivating the E3 ligase of the Hdm2/HdmX complex. This specific mechanism of action allows the good biological activity of cyclotide MCo-52-2 against several human cancer cell lines in a p53-independent fashion. The compounds also can be formulated with intestinal absorption enhancers (such as sodium salcaprozate, SNAC) to enhance efficacy.

Biophysical characterization of selected cyclotides. has been performed using NMR. The solution of structure of the complex formed by cyclotide MCo-52-2 and the RING domain of HdmX (FIG. 2B) reveals that the interaction is mostly mediated by cyclotide residues located in loops 1 and 5 interacting with the C-terminal beta-strand of the HdmX RING domain. This interaction prevents the RING-mediated dimerization of HdmX with Hdm2 hence inhibiting its E3 ligase activity.

Applicant developed a potent inhibitor of Hdm2-Hdmx E3-ligase activity active in vitro and in vivo models of CRC. Bioactive cyclotides highly specific for antagonizing Hdm2-HdmX E3-ligase, and for eliciting both p53-dependent and p53-independent cytotoxicity in CRC cancer cells were made. The use of the cyclotide scaffold enables these peptide antagonists to have the required increased stability, cellular membrane penetration, proteolysis resistance and serum clearance, thus establishing cyclotides as a new and promising platform for therapeutic drug development.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout this disclosure, various publication are referenced by a citation, the full bibliographic citation for each are provided immediately preceding the claims.

### REFERENCES

1. Siegel, R. L., Miller, K. D., Fuchs, H. E., and Jemal, A. (2021) Cancer Statistics, 2021, CA Cancer J. Clin. 71: 7-33.
2. Hou, H., Sun, D., and Zhang, X. (2019) The role of MDM2 amplification and overexpression in therapeutic resistance of malignant tumors, Cancer Cell Int. 19: 216.
3. Yu, D. H., Xu, Z. Y., Mo, S. W., Yuan, L., Cheng, X. D., and Qin, J. J. (2020) Targeting MDMX for Cancer Therapy: Rationale, Strategies, and Challenges, Front. Oncol. 10.
4. Shmueli, A., and Oren, M. (2004) Regulation of p53 by Mdm2: fate is in the numbers, Mol. Cell 13: 4-5.
5. Stommel, J. M., and Wahl, G. M. (2004) Accelerated MDM2 auto-degradation induced by DNA-damage kinases is required for p53 activation, EMBO J. 23: 1547-1556.
6. Riley, M. F., and Lozano, G. (2012) The Many Faces of MDM2 Binding Partners, Genes Cancer 3: 226-239.
7. Jiang, X. J., Kim, K. J., Ha, T., and Lee, S. H. (2016) Potential Dual Role of Activating Transcription Factor 3 in Colorectal Cancer, Anticancer Res. 36: 509-516.
8. Bullock, M. D., Bruce, A., Sreekumar, R., Curtis, N., Cheung, T., Reading, I., Primrose, J. N., Ottensmeier, C., Packham, G. K., Thomas, G., and Mirnezami, A. H. (2013) FOXO3 expression during colorectal cancer progression: biomarker potential reflects a tumour suppressor role, Br. J. Cancer 109: 387-394.
9. Liu, Y., Ao, X., Ding, W., Ponnusamy, M., Wu, W., Hao, X., Yu, W., Wang, Y., Li, P., and Wang, J. (2018) Critical role of FOXO3a in carcinogenesis, Mol. Cancer 17: 104.
10. Soong, R., Shah, N., Peh, B. K., Chong, P. Y., Ng, S. S., Zeps, N., Joseph, D., Salto-Tellez, M., Iacopetta, B., and Ito, Y. (2009) The expression of RUNX3 in colorectal cancer is associated with disease stage and patient outcome, Br. J. Cancer 100: 676-679.
11. Xue, J., Wu, X. L., Qu, M., Guo, F., Han, L., Sun, G. Y., Yuan, Z. L., Fan, S., and Li, T. (2020) RUNX3 Inhibits the Invasion and Metastasis of Human Colon Cancer HT-29 Cells by Upregulating MMP-2/9, Evid. Based Complement. Alternat. Med. 2020.
12. Yan, H., Hu, K., Wu, W., Li, Y., Tian, H., Chu, Z., Koeffler, H. P., and Yin, D. (2016) Low Expression of DYRK2 (Dual Specificity Tyrosine Phosphorylation Regulated Kinase 2) Correlates with Poor Prognosis in Colorectal Cancer, PLoS One 11: e0159954.
13. Ito, D., Yogosawa, S., Mimoto, R., Hirooka, S., Horiuchi, T., Eto, K., Yanaga, K., and Yoshida, K. (2017) Dual-specificity tyrosine-regulated kinase 2 is a suppressor and potential prognostic marker for liver metastasis of colorectal cancer, Cancer Sci. 108: 1565-1573.
14. Jung, H. Y., Wang, X., Jun, S., and Park, J. I. (2013) Dyrk2-associated EDD-DDB1-VprBP E3 ligase inhibits telomerase by TERT degradation, J. Biol. Chem. 288: 7252-7262.
15. Hiyama, E., and Hiyama, K. (2007) Telomere and telomerase in stem cells, Br. J. Cancer 96: 1020-1024.
16. Tandon, V., de la Vega, L., and Banerjee, S. (2020) Emerging roles of DYRK2 in cancer, J. Biol. Chem.
17. D'Orazi, G., Rinaldo, C., and Soddu, S. (2012) Updates on HIPK2: a resourceful oncosuppressor for clearing cancer, J. Exp. Clin. Cancer Res. 31: 63.
18. Gu, J., Kawai, H., Nie, L., Kitao, H., Wiederschain, D., Jochemsen, A. G., Parant, J., Lozano, G., and Yuan, Z. M. (2002) Mutual dependence of MDM2 and MDMX in their functional inactivation of p53, J. Biol. Chem. 277: 19251-19254.
19. Linares, L. K., Hengstermann, A., Ciechanover, A., Muller, S., and Scheffner, M. (2003) HdmX stimulates Hdm2-mediated ubiquitination and degradation of p53, Proc. Natl. Acad. Sci. U.S.A 100: 12009-12014.
20. Tanimura, S., Ohtsuka, S., Mitsui, K., Shirouzu, K., Yoshimura, A., and Ohtsubo, M. (1999) MDM2 interacts with MDMX through their RING finger domains, FEBS Lett. 447: 5-9.
21. Kostic, M., Matt, T., Martinez-Yamout, M. A., Dyson, H. J., and Wright, P. E. (2006) Solution structure of the Hdm2 C2H2C4 RING, a domain critical for ubiquitination of p53, J. Mol. Biol. 363: 433-450.
22. Linke, K., Mace, P. D., Smith, C. A., Vaux, D. L., Silke, J., and Day, C. L. (2008) Structure of the MDM2/MDMX RING domain heterodimer reveals dimerization is required for their ubiquitylation in trans, Cell Death Differ 15: 841-848.
23. Sanchez-Carbayo, M., Socci, N. D., Lozano, J. J., Haab, B. B., and Cordon-Cardo, C. (2006) Profiling bladder cancer using targeted antibody arrays, Am. J. Pathol. 168: 93-103.
24. Chaudhuri, D., Aboye, T., and Camarero, J. A. (2019) Using backbone-cyclized Cys-rich polypeptides as molecular scaffolds to target protein-protein interactions, Biochem J 476: 67-83.
25. Poth, A. G., Colgrave, M. L., Lyons, R. E., Daly, N. L., and Craik, D. J. (2011) Discovery of an unusual biosynthetic origin for circular proteins in legumes, Proc. Natl. Acad. Sci. U.S.A 108: 10127-10132.
26. Camarero, J. A., and Campbell, M. J. (2019) The Potential of the Cyclotide Scaffold for Drug Development, Biomedicines 7.
27. Austin, J., Kimura, R. H., Woo, Y. H., and Camarero, J. A. (2010) In vivo biosynthesis of an Ala-scan library based on the cyclic peptide SFTI-1, Amino Acids 38: 1313-1322.
28. Li, Y., Bi, T., and Camarero, J. A. (2015) Chemical and biological production of cyclotides, Adv. Bot. Res. 76: 271-303.
29. Saether, O., Craik, D. J., Campbell, I. D., Sletten, K., Juul, J., and Norman, D. G. (1995) Elucidation of the primary and three-dimensional structure of the uterotonic polypeptide kalata B1, Biochemistry 34: 4147-4158.
30. Contreras, J., Elnagar, A. Y., Hamm-Alvarez, S. F., and Camarero, J. A. (2011) Cellular uptake of cyclotide MCoTI-I follows multiple endocytic pathways, J. Control. Release 155: 134-143.
31. Ji, Y., Majumder, S., Millard, M., Borra, R., Bi, T., Elnagar, A. Y., Neamati, N., Shekhtman, A., and Camarero, J. A. (2013) In vivo activation of the p53 tumor suppressor pathway by an engineered cyclotide, J. Am. Chem. Soc. 135: 11623-11633.
32. Slazak, B., Kapusta, M., Malik, S., Bohdanowicz, J., Kuta, E., Malec, P., and Goransson, U. (2016) Immunolocalization of cyclotides in plant cells, tissues and organ supports their role in host defense, Planta 244: 1029-1040.
33. Garcia, A. E., and Camarero, J. A. (2010) Biological activities of natural and engineered cyclotides, a novel molecular scaffold for peptide-based therapeutics, Curr. Mol. Pharmacol. 3: 153-163.
34. Aboye, T., Meeks, C. J., Majumder, S., Shekhtman, A., Rodgers, K., and Camarero, J. A. (2016) Design of a MCoTI-Based Cyclotide with Angiotensin (1-7)-Like Activity, Molecules 21: 152.
35. Aboye, T. L., Ha, H., Majumder, S., Christ, F., Debyser, Z., Shekhtman, A., Neamati, N., and Camarero, J. A. (2012) Design of a novel cyclotide-based CXCR4 antagonist with anti-human immunodeficiency virus (HIV)-1 activity, J. Med. Chem. 55: 10729-10734.
36. Kimura, R. H., Steenblock, E. R., and Camarero, J. A. (2007) Development of a cell-based fluorescence resonance energy transfer reporter for Bacillus anthracis lethal factor protease, Anal. Biochem. 369: 60-70.
37. Nicolae, M., Mangul, S., Mandoiu, II, and Zelikovsky, A. (2011) Estimation of alternative splicing isoform frequencies from RNA-Seq data, Algorithms Mol. Biol. 6: 9.
38. Wang, S., Sun, W., Zhao, Y., McEachern, D., Meaux, I., Barriere, C., Stuckey, J. A., Meagher, J. L., Bai, L., Liu, L., Hoffman-Luca, C. G., Lu, J., Shangary, S., Yu, S., Bernard, D., Aguilar, A., Dos-Santos, O., Besret, L., Guerif, S., Pannier, P., Gorge-Bernat, D., and Debussche, L. (2014) SAR405838: an optimized inhibitor of MDM2-p53 interaction that induces complete and durable tumor regression, Cancer Res. 74: 5855-5865.

1. An isolated cyclotide polypeptide having the amino acid sequence SEQ ID NO: 1: 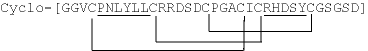
and variants thereof, wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline).

2. The isolated cyclotide polypeptide of claim 1, further comprising SEQ ID NO: 2: 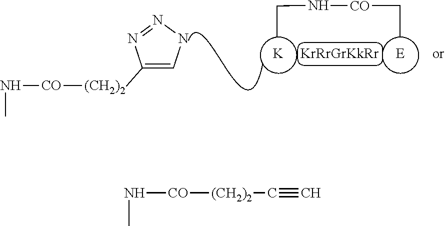
covalently attached to K at amino acid 15.

3. The isolated cyclotide polypeptide of claim 1, wherein the cyclotide has a structure selected from: 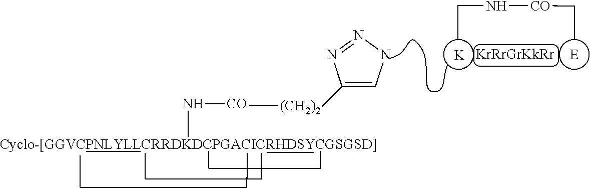
(SEQ ID NOS 2 and 3, respectively, in order of appearance) and variants thereof,
wherein amino acids 31 and 34 are optionally Thr, Asn, Gln, Asp, Glu, Lys, Arg; or wherein amino acid 3 is optionally Val, Ile, Leu, Phe, Tye, Trp, Cha (cyclohexylalanine), Chg (cyclohexylglycine), Phg (phenylglycine), Nle (norleucine), Tle (terleucine), or Nva (norvaline),
wherein the MCo-52-2 cyclotide backbone is joined to the cTAT peptide by a linker comprising —(O—CH₂CH₂—)₃, and
wherein capital letters indicate an L-amino acid and lower case letters indicate D amino acids;
or 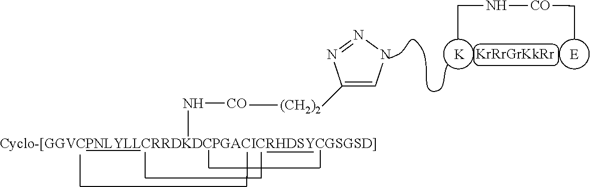
(SEQ ID NOS 2 and 3, respectively, in order of appearance) wherein the MCo-52-2 cyclotide backbone is joined to the cTAT peptide by a linker comprising —(O—CH₂CH₂—)₃, and
wherein capital letters indicates an L-amino acid and lower case letters indicate D amino acids.

4. A plurality of cyclotides of claim 1, optionally wherein the amino acid sequences of the plurality are the same or different from each other or further optionally further comprising a carrier, further optionally wherein the carrier is a pharmaceutically acceptable carrier and/or an additional anti-cancer therapy.

5-8. (canceled)

9. An isolated polynucleotide encoding the cyclotide of claim 1 or a complement thereto, and optionally a carrier, further optionally a pharmaceutically acceptable carrier.

10-13. (canceled)

14. A vector or an isolated host cell comprising the isolated polynucleotide of claim 9, optionally wherein the cell is a eukaryotic cell or a prokaryotic cell.

15. An isolated host cell comprising the cyclotide of claim 1, optionally wherein the cell is a eukaryotic cell or a prokaryotic cell.

16. (canceled)

17. A method for producing a cyclotide, comprising expressing the polynucleotide of claim 9 that encodes the cyclotide in a host cell, under conditions to express the polynucleotide and optionally chemically modifying the cyclotide, and optionally further purifying the cyclotide.

18. (canceled)

19. A method to inhibit the binding of Hdm2 or HdmX to binding partners, comprising contacting Hdm2 or HdmX with an effective amount of the cyclotide of claim 3, thereby inhibiting the binding of Hdm2 or HdmX to its binding partner, optionally wherein the ring domains of Hdm2 or HdmX are inhibited or wherein the binding partner of Hdm2 is selected from p53, Hdm2 or HdmX.

20-21. (canceled)

22. A method to inhibit E3 ligase activity, comprising contacting the E3 ligase with an effective amount of the cyclotide of claim 3, thereby inhibiting E3 ligase activity.

23. A method to stabilize an enzyme or peptide regulated by E3 ligase, comprising contacting the enzyme or peptide with an effective amount of the cyclotide of claim 3, thereby stabilizing the enzyme or peptide regulated by E3 ligase, optionally wherein the enzyme or peptide is selected from a peptide shown in FIG. 8, p53, ATF3, FOXO3a and RUNX3; and kinases DYRK2 and HIPK2 or wherein the enzyme or peptide is stabilized by reducing or eliminating degradation of the enzyme or peptide.

24-27. (canceled)

28. The method of inhibiting the growth of a cancer cell, comprising contacting the cancer cell with an effective amount of the cyclotide of claim 3, thereby inhibiting the growth of the cancer cell, optionally wherein the cancer cell is selected from a colon cancer cell, a leukemia cell, a lung cancer cell, a small cell lung cancer cell, a pancreatic cancer cell, an ovarian cancer cell, a prostate cancer cell or a breast cancer cell or wherein the contacting is in vitro or in vivo.

29-31. (canceled)

32. A method to treat cancer or tumor in a subject in need thereof, comprising administering to the subject an effective amount of the cyclotide of claim 3, thereby treating the cancer or the tumor, optionally wherein the tumor is a solid tumor or wherein the cancer is a colorectal cancer, a colon cancer, a leukemia, a lung cancer, a small cell lung cancer, a pancreatic cancer, an ovarian cancer, a prostate cancer or a breast cancer.

33. A method to induce an anti-cancer immune response in a subject having a tumor or cancer in need thereof, comprising administering to the subject an effective amount of the cyclotide of claim 3, thereby thereby inducing an anti-cancer immune response in a subject having the tumor or cancer, optionally wherein the tumor is a solid tumor or wherein the cancer is selected from a colorectal cancer, a colon cancer, a leukemia, a lung cancer, a small cell lung cancer, a pancreatic cancer, an ovarian cancer, a prostate cancer or a breast cancer.

34-40. (canceled)

41. A kit comprising one or more of the cyclotide of claim 1 or a polynucleotide encoding the cyclotide.


SEQUENCE LISTING (9 sequences)

SEQ ID NO: 1 - protein, 34 aa, synthetic construct
  DISULFID 4..21: note = Intrachain disulfide bond
  DISULFID 11..23: note = Intrachain disulfide bond
  DISULFID 17..29: note = Intrachain disulfide bond
GGVCPNLYLLCRRDSDCPGACICRHDSYCGSGSD

SEQ ID NO: 2 - protein, 12 aa, synthetic construct
  SITE 1: note = Residue attached to azido-3x polyethylene glycol acid linker
  SITE 3: note = D-amino acid
  SITE 5: note = D-amino acid
  SITE 7: note = D-amino acid
  SITE 9: note = D-amino acid
  SITE 11: note = D-amino acid
KKRRRGRKKRRE

SEQ ID NO: 3 - protein, 34 aa, synthetic construct
  SITE 15: note = Residue attached to 3-(1,2,3-triazol-4-yl)propanamide linker
  DISULFID 4..21: note = Intrachain disulfide bond
  DISULFID 11..23: note = Intrachain disulfide bond
  DISULFID 17..29: note = Intrachain disulfide bond
GGVCPNLYLLCRRDKDCPGACICRHDSYCGSGSD

SEQ ID NO: 4 - protein, 34 aa, synthetic construct; identical to SEQ ID NO: 3
  SITE 15: note = Residue attached to pent-4-ynamide
  DISULFID 4..21: note = Intrachain disulfide bond
  DISULFID 11..23: note = Intrachain disulfide bond
  DISULFID 17..29: note = Intrachain disulfide bond

SEQ ID NO: 5 - DNA, 105 bp, synthetic construct
  source 1..105: mol_type = other DNA
  misc_difference 3: note = a, c, t, g, unknown or other
  misc_difference 6: note = a, c, t, g, unknown or other
  misc_difference 9: note = a, c, t, g, unknown or other
  misc_difference 15: note = a, c, t, g, unknown or other
  misc_difference 21: note = a, c, t, g, unknown or other
  misc_difference 27: note = a, c, t, g, unknown or other
  misc_difference 30: note = a, c, t, g, unknown or other
  misc_difference 36: note = a, c, t, g, unknown or other
  misc_difference 39: note = a, c, t, g, unknown or other
  misc_difference 54: note = a, c, t, g, unknown or other
  misc_difference 57: note = a, c, t, g, unknown or other
  misc_difference 60: note = a, c, t, g, unknown or other
  misc_difference 72: note = a, c, t, g, unknown or other
  misc_difference 81: note = a, c, t, g, unknown or other
  misc_difference 90: note = a, c, t, g, unknown or other
  misc_difference 93: note = a, c, t, g, unknown or other
  misc_difference 96: note = a, c, t, g, unknown or other
  misc_difference 99: note = a, c, t, g, unknown or other
  misc_difference 102: note = a, c, t, g, unknown or other
ggnggngtntgyccnaayytntayytnytntgymgnmgngayaargaytgyccnggngcntgyathtgymgncaygaywsntaytgyggnwsnwsnggnwsngay

SEQ ID NO: 6 - protein, 7 aa, synthetic construct
MENLYFQ

SEQ ID NO: 7 - DNA, 44 bp, synthetic construct
  source 1..44: mol_type = other DNA
aaacatatggaaaacctgtacttccagtgcggttctggttctgg

SEQ ID NO: 8 - DNA, 20 bp, synthetic construct
  source 1..20: mol_type = other DNA
gattgccatgccggtcaagg

SEQ ID NO: 9 - protein, 34 aa, synthetic construct
  SITE 34: note = Residue attached to a thiol group
CGSGSDGGVCPNLYLLCRRDSDCPGACICRHDSY